(19) United States
(12) Patent Application Publication
(10) Pub. No.: US 2024/0241541 A1
(43) Pub. Date: Jul. 18, 2024

(54) OPTICAL COMPONENTS AND ASSEMBLY TECHNIQUES

(71) Applicant: Oura Health Oy, Oulu (FI)

(72) Inventor: Jukka Tapani Mäkinen, Oulu (FI)

(21) Appl. No.: 18/410,866

(22) Filed: Jan. 11, 2024

Related U.S. Application Data
(60) Provisional application No. 63/479,945, filed on Jan. 13, 2023.
(60) Provisional application No. 63/586,637, filed on Sep. 29, 2023.

(51) Int. Cl. G06F 1/16; G02B 6/42

(52) U.S. Cl. CPC G06F 1/163; G02B 6/4214; G02B 6/4245; G02B 6/4281

(57) ABSTRACT

Methods, systems, and devices for manufacturing a device are described. The manufacturing system may assemble a plurality of optical components onto an inner circumference of an inner housing member of a wearable ring device and align the plurality of optical components with a plurality of apertures of the inner housing member. In some cases, the manufacturing system may position a plurality of optoelectronic components affixed to a flexible printed circuit board onto the inner circumference after the plurality of optical components are assembled onto the inner circumference. The manufacturing system may adhere the plurality of optoelectronic components to the plurality of optical components such that the plurality of optical components are aligned with the plurality of optoelectronic components.

### CROSS REFERENCE

The present application for patent claims the benefit of U.S. Provisional Patent Application No. 63/479,945 by MAKINEN, entitled “ARRAY OPTICS AND FABRICATION,” filed Jan. 13, 2023, and U.S. Provisional Patent Application No. 63/586,637 by MAKINEN, entitled “OPTICAL COMPONENTS AND ASSEMBLY TECHNIQUES,” filed Sep. 29, 2023, each of which is assigned to the assignee thereof, and each of which is expressly incorporated by reference herein.

### FIELD OF TECHNOLOGY

The following relates to wearable devices and data processing, including optical components and assembly techniques.

### BACKGROUND

Some wearable devices may be configured to collect physiological data from users, including heart rate, motion data, temperature data, photoplethysmogram (PPG) data, and the like. In some cases, some wearable devices may perform various actions, such as providing certain health insights to users, based on acquired physiological data in order to assist the user with improving their overall health.

Methods for manufacturing wearable devices may include fabricating each individual component of the wearable device. However, techniques used in such manufacturing processes may lead to irregularities in shape, may be costly, may be difficult to position to create a consistent product, among other potential deficiencies.

### BRIEF DESCRIPTION OF THE DRAWINGS

FIG. 1 illustrates an example of a system that supports optical components and assembly techniques in accordance with aspects of the present disclosure.

FIG. 2 illustrates an example of a system that supports optical components and assembly techniques in accordance with aspects of the present disclosure.

FIGS. 3A and 3B shows examples of a manufacturing system that supports optical components and assembly techniques in accordance with aspects of the present disclosure.

FIG. 4 shows an example of a manufacturing system that supports optical components and assembly techniques in accordance with aspects of the present disclosure.

FIG. 5 shows an example of a manufacturing system that supports optical components and assembly techniques in accordance with aspects of the present disclosure.

FIG. 6 shows an example of a manufacturing system that supports optical components and assembly techniques in accordance with aspects of the present disclosure.

FIGS. 7A, 7B, and 7C shows examples of manufacturing systems that supports optical components and assembly techniques in accordance with aspects of the present disclosure.

FIG. 8 shows an example of wearable device diagrams that supports optical components and assembly techniques in accordance with aspects of the present disclosure.

FIG. 9 shows an example of wearable device diagrams that supports optical components and assembly techniques in accordance with aspects of the present disclosure.

FIG. 10 shows an example of wearable device diagrams that supports optical components and assembly techniques in accordance with aspects of the present disclosure.

FIG. 11 shows an example of a flowchart that supports optical components and assembly techniques in accordance with aspects of the present disclosure.

FIG. 12 shows an example of a flowchart that supports optical components and assembly techniques in accordance with aspects of the present disclosure.

### DETAILED DESCRIPTION

An individual may use a wearable device (e.g., a wearable ring device) to collect, monitor, and track physiological data of the individual based on sensor measurements of the wearable ring device. Examples of physiological data may include temperature data, heart rate data, photoplethysmography (PPG) data, and the like. The physiological data collected, monitored, and tracked via the wearable ring device may be used to gain health insights about the user.

In some cases, one or more sensors of the wearable ring device may be located between an inner housing of the wearable ring device and an outer housing of the wearable ring device. Separate optical components (e.g., lenses, reflectors, prisms, microprisms, micro lenses arrays, angular filters, and the like) may be included in the wearable ring device to enhance the functionality of the one or more sensor optoelectronic components (e.g., components such as light emitting elements and light detecting elements). In particular, the optical components may direct light to the one or more photodetectors, manipulate the field of view of the one or more photodetectors, and improve the internal stray light suppression.

However, integrating the optical components into the wearable ring device with adequate accuracy and robustness may be challenging due to the size of the components and the scale of the integration. The optical components may need to be properly positioned within the wearable ring devices to align with the one or more sensor optoelectronic components, resulting in manufacturing complexities or, if misaligned, manufacturing deformities. In such cases, manufacturing the optical components to adhere to the wearable ring device may be expensive and time consuming due to the need to accurately place the optical components onto or within the wearable ring device. Further, the optical components may provide an even distribution of light and/or direct light in a single direction (e.g., the center of the finger). In this regard, without modifying the emission pattern (e.g., directionality, depth of penetration, distribution, etc.), the internal stray light suppression may increase, the quality of the signal may decrease, and the power consumption may increase.

In this regard, inefficiently manufacturing optical components may result in inaccurate placement of the optical components that may result in unreliable physiological measurements. Further, a size of the optical components may depend on a size of the wearable ring device and/or a size of the one or more sensors, resulting in additional manufacturing complexities and costs to manufacture each optical component separately. In some cases, inaccurately manufacturing optical components may result in manufacturing deformities that may require additional manufacturing to remove the deformities (e.g., polishing, etching, sanding), thereby increasing the cost of production and increasing the waste associated with discarding the inaccurately manufactured optical components.

Accordingly, aspects of the present disclosure may support a manufacturing process that reduces manufacturing complexities and deformities. In some cases, the functionality of the optoelectronic components may be enhanced by customizing the optical components. Customization may include a shape of the optical components, a reflective material on the optical components, microprisms, and the like that may increase a collection area of the signal, directionally control the light, and increase or decrease an emitter beam width, thereby improving the overall signal quality to increase the reliability of PPG measurements.

For example, the customization may modify the emission pattern of light emitted by the optical components such that the emitter beam width and directionality may be adjusted to target a specific location (e.g., physiological structure within the finger), and the photodetector field of view may be modified to enhance the signal quality. That is, an intersection of the light source emission pattern and the photo detector field of view may target a certain physiology within the finger, and the intersection may be located closer to the photodetector, thereby improving the signal quality and decreasing the overall power consumption. By adjusting the intersection of the light source emission pattern and the photodetector field of view, the array optics may offer better control over what is the exact target location inside the finger and how much signal can be obtained for a more efficient measurement.

The manufacturing system may deposit the optical components onto an inlet piece of the wearable device. The one or more sensors of a flexible printed circuit board (PCB) may be positioned onto the optical components after the optical components are deposited onto the wearable device. In such cases, the method of manufacturing may include assembling a substrate (e.g., flexible foil) that includes the optical components to an inlet piece of the wearable device before adhering the flexible PCB to the optical components. For example, the substrate may be wrapped around the inlet piece to attach the optical components to the correct position over the inlet apertures, and then the flexible PCB may then be adhered to the ring inlet after the optical components are adhered. The one or more sensors may be an example of optoelectronic components that include a set of light emitting components and a set of light detecting components.

In such cases, the optical components and the one or more sensors may be aligned, thus enabling the wearable ring device to be fabricated with a reduced likelihood of manufacturing deformities and enabling the manufacturing process to be completed with increased efficiency, increased speed, and reduced manufacturing complexity. Accordingly, manufacturing the optical components to be aligned with the one or more sensors may result in high-volume production of optics arrays (e.g., optical components) manufactured on the inner ring inlet and a low-cost solution to the assembly. That is, enhanced optical functionality by depositing the optical components onto the inner ring inlet and then aligning the optical components with the one or more sensors during the manufacturing process may introduce increased signal quality and stronger signals, thereby reducing the power consumption and increasing the battery life of the wearable ring device.

Although the examples described herein are related to manufacturing a wearable ring device, it should be understood that the described techniques and devices may be applied to manufacturing processes related to other devices or items, such as other wearables (e.g., watches, bands), or other electronic devices that are not considered wearables.

Aspects of the disclosure are initially described in the context of systems supporting physiological data collection from users via wearable devices. Aspects are then described in the context of manufacturing systems and wearable device diagrams. Aspects of the disclosure are further illustrated by and described with reference to apparatus diagrams, system diagrams, and flowcharts that relate to optical components and assembly techniques.

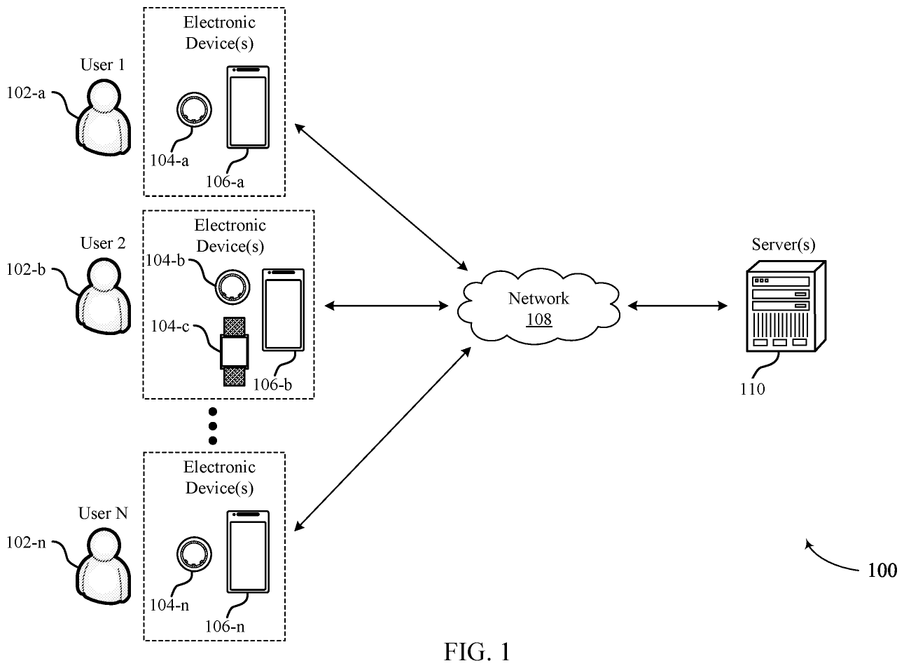

FIG. 1 illustrates an example of a system 100 that supports optical components and assembly techniques in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the car, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for a manufacturing process (e.g., and system) that reduces manufacturing complexities and deformities. For example, a ring 104, such as the ring 104-a, the ring 104-b, the ring 104-n, or any combination thereof, may include an inner housing (e.g., inner housing member) and an outer housing (e.g., outer housing member), where one or more sensors are positioned between the inner housing and the outer housing. In such cases, a manufacturing process may include, at least, depositing a plurality of optical components onto an inner ring inlet of the ring 104 and then positioning a plurality of optoelectronic components (e.g., one or more sensors) onto the plurality of optical components to align the plurality of optoelectronic components with the plurality of optical components, thereby securing the one or more sensors in place without producing manufacturing deformities and inconsistencies.

For example, a manufacturing system may assemble the plurality of optical components onto an inner circumference (e.g., the inner ring inlet) of an inner housing member of the ring 104. The plurality of optical components may be aligned with a plurality of apertures of the inner ring inlet. In some cases, the plurality of optical components may include a first type of optical components configured to manipulate a light emission direction or a light detection direction and a second type of optical components configured to at least partially seal the plurality of apertures. The manufacturing system position the plurality of optoelectronic components of the flexible PCB onto the inner circumference after assembling the plurality of optical components onto the inner circumference of the ring 104. The plurality of optoelectronic components may be an example of one or more sensors that include a set of light emitting components and a set of light detecting components. The manufacturing system may adhere the plurality of optoelectronic components to the plurality of optical components such that the plurality of optical components are aligned with the plurality of optoelectronic components. In some cases, the first type of optical components may be positioned between the plurality of optoelectronic components and the second type of optical components.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

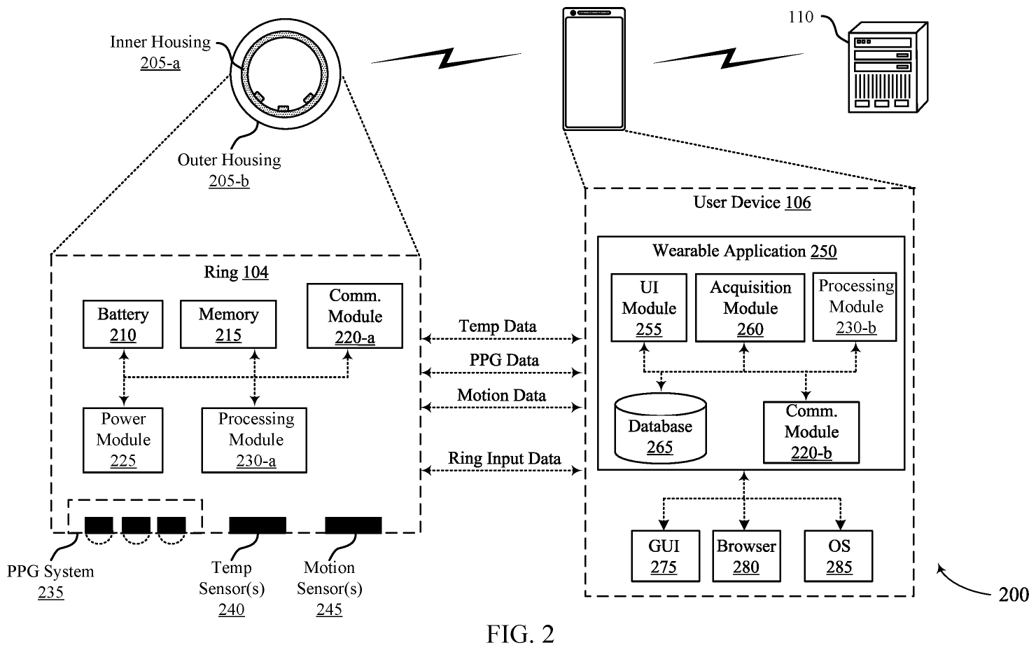

FIG. 2 illustrates an example of a system 200 that supports optical components and assembly techniques in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for a manufacturing process (e.g., and system) that reduces manufacturing complexities and inconsistencies. For example, as described previously, the ring 104 may include an inner housing 205-a (e.g., inner housing member) and an outer housing 205-b (e.g., outer housing member), where one or more sensors, such as a PPG system 235, one or more temperature sensors 240, one or more motion sensors 245, or any combination thereof, are positioned between the inner housing 205-a and the outer housing 205-b. In such cases, a manufacturing process may include, at least, a positioning process to enable the plurality of optical components to be aligned with the plurality of optoelectronic components (e.g., including the one or more sensors) after the plurality of optical components are assembled onto the ring 104.

For example, a manufacturing system may assemble the plurality of optical components onto an inner circumference of an inner housing member of the ring 104 and align the plurality of optical components with a plurality of apertures of the inner housing member of the ring 104. The plurality of optical components may include a first type of optical components configured to manipulate a light emission direction or a light detection direction and a second type of optical components configured to at least partially seal the plurality of apertures.

The manufacturing system may position the one or more sensors of a flexible PCB onto the inner circumference of the inner housing member of the ring 104 after assembling the plurality of optical components onto the inner circumference. The manufacturing system may adhere the plurality of optoelectronic components to the plurality of optical components such that the plurality of optical components are aligned with the plurality of optoelectronic components and the first type of optical components are positioned between the plurality of optoelectronic components and the second type of optical components.

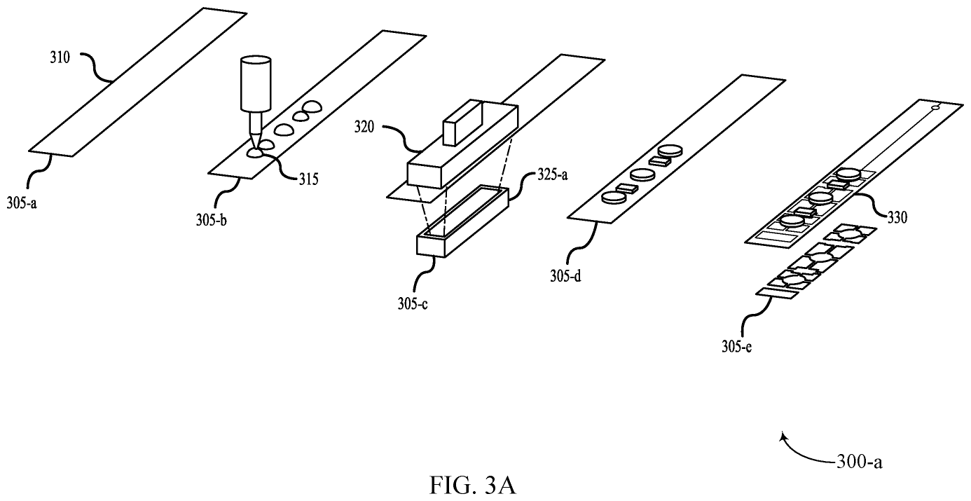

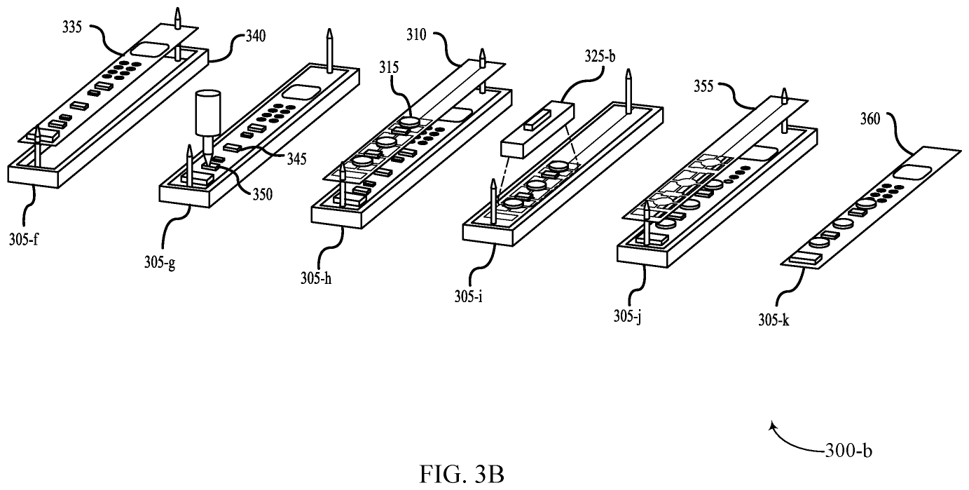

FIG. 3 shows an example of a manufacturing system 300 that supports optical components and assembly techniques in accordance with aspects of the present disclosure. The manufacturing system 300 may implement, or be implemented by, the system 100 and the system 200. In particular, the manufacturing system 300 illustrates a manufacturing process to manufacture an array optics assembly 360, as described with reference to FIGS. 1 and 2.

In some examples (e.g., in a first part of the manufacturing process as described with reference to FIG. 3A), a transparent carrier foil, that may also be referred to as an alignment substrate 310, may be obtained (e.g., by the manufacturing system 300) for processing step 305-a. The alignment substrate 310 may include a transparent material, a flexible material, or both.

At processing step 305-b, a plurality of optical components 315 may be deposited onto the alignment substrate 310. For example, the optical material of the plurality of optical components 315 may be dispensed (e.g., deposited) onto the alignment substrate 310. In some cases, the manufacturing system 300 may deposit the optical material of the plurality of optical components 315 linearly along the alignment substrate 310. The optical material of the plurality of optical components 315 may include an ultraviolet (UV) curable material, a moldable material, or both.

In some examples, the plurality of optical components 315, may be UV cured (e.g., by the manufacturing system 300) with a mold 320 as described with reference to processing step 305-c. For example, the manufacturing system 300 may position a portion of the alignment substrate 310 through the mold 320 after the material of the plurality of optical components 315 is deposited. In such cases, the manufacturing system 300 may form a shape of the plurality of optical components 315.

In other examples, the manufacturing system 300 may position a portion of the alignment substrate 310 through the mold 320 and inject a material of the plurality of optical components 315 into the mold 320. In such examples, instead of depositing the material of the plurality of optical components 315 onto the alignment substrate 310 prior to positioning the alignment substrate 310 within the mold 320, the manufacturing system 300 may deposit the material of the plurality of optical components 315 onto the alignment substrate 310 after positioning the alignment substrate 310 within the mold 320.

At processing step 305-c, the manufacturing system 300 may apply a light source 325-a to the plurality of optical components 315 to create a seal between the plurality of optical components 315 and the alignment substrate 310. For example, the manufacturing system 300 may apply UV light to the UV curable material of the plurality of optical components 315. In such cases, the plurality of optical components 315 may be integrated into the alignment substrate 310 such that the interface between the plurality of optical components 315 and the alignment substrate 310 is removed and the structure (e.g., including the alignment substrate 310 and the plurality of optical components 315) is sealed.

At processing step 305-d, the manufacturing system 300 may release the plurality of optical components 315 from the mold after the plurality of optical components 315 are integrated into the alignment substrate 310. In such cases, the manufacturing system 300 may form a shape of the plurality of optical components 315.

At processing step 305-e, the alignment substrate 310 may be perforated for an optics assembly, as described with reference to FIG. 3A. In such cases, the manufacturing system 300 may perforate the alignment substrate 310 to form perforations 330 that surround the plurality of optical components 315. The plurality of optical components 315 may be maintained on the alignment substrate throughout the manufacturing process such that an array optics assembly may be created in a single process and added to ring components (e.g., including the optical components, as described herein). For example, the manufacturing system 300 may create holes through the alignment substrate 310 while maintaining a distance between each of the plurality of optical components 315.

In some examples (e.g., in a second part of the manufacturing process as described with reference to FIG. 3B), a ring electronics assembly, that may also be referred to as flexible PCB 335, may be positioned (e.g., by the manufacturing system 300) into an assembly jig 340 (e.g., alignment jig). For example, at processing step 305-f, the manufacturing system 300 may position the alignment substrate 310 within the assembly jig 340. The assembly jig 340 may include a first arm and a second arm such that the alignment substrate 310 is aligned with the flexible PCB 335 within the assembly jig 340.

At processing step 305-g, the manufacturing system 300 may dispense a transparent glue 350 onto a plurality of optoelectronic components 345 of the flexible PCB 335. The plurality of optoelectronic components 345 may include a set of light emitting components and a set of light detecting components. As described herein, an optical interface between the plurality of optical components 315 and the plurality of optoelectronic components 345 may be made by the transparent glue 350.

At processing step 305-h, the manufacturing system 300 may position, using one or more alignments features of the alignment substrate 310, the plurality of optical components 315 onto the plurality of optoelectronic components 345. In such cases, the plurality of optical components 315 may be assembled on top of the plurality of optoelectronic components 345.

In some cases, the manufacturing system 300 may fix the plurality of optical components 315 to the plurality of optoelectronic components 345 by UV curing. In such cases, at processing step 305-i, the manufacturing system 300 may adhere the alignment substrate 310 to the plurality of optoelectronic components 345 such that the alignment substrate 310 is disposed between the plurality of optoelectronic components 345 and the plurality of optical components 315. The plurality of optical components 315 may be aligned with the plurality of optoelectronic components 345.

In some examples, the manufacturing system 300 may apply a light source 325-b to the plurality of optoelectronic components 345 to create a seal between the plurality of optoelectronic components 345 and the plurality of optical components 315 after adhering the alignment substrate 310 to the plurality of optoelectronic components 345. For example, the manufacturing system 300 may apply UV light to the plurality of optoelectronic components 345. In such cases, the plurality of optical components 315 may be integrated onto the plurality of optoelectronic components 345 such that the interface between the plurality of optical components 315 and the plurality of optoelectronic components 345 is removed and the structure (e.g., including the alignment substrate 310, the plurality of optical components 315, and the plurality of optoelectronic components 345) is sealed.

At processing step 305-j, the alignment substrate 310 may be removed. In such cases, the manufacturing system 300 may remove a non-adhered portion 355 of the alignment substrate 310 from the flexible PCB 335 after adhering the alignment substrate 310 to the plurality of optoelectronic components 345. The alignment substrate 310 may initially be utilized to increase a thickness of the structure and increase the durability throughout the manufacturing process.

At processing step 305-k, the array optics assembly 360 to be included in the wearable ring device may include a plurality of optoelectronic components 345 including the set of light emitting components and the set of light detecting components positioned on the flexible PCB 335, a plurality of optical components 315 adhered to the plurality of optoelectronic components 345, and at least a portion of the alignment substrate 310 disposed between the plurality of optoelectronic components 345 and the plurality of optical components 315.

In some cases, a dome structure that encompasses the light detecting components of the ring structure, as described with reference to FIGS. 1 and 2, may be replaced with separate, optical pieces (e.g., optical components 315). The optical components 315 may include a dome shape, a mirror coated reflector material, or both. In such cases, the plurality of optical components 315 may include a reflective material that may coat the outer surface of the plurality of optical components 315. The reflective material may increase a surface area of light collection for the light detecting components. For example, the light detecting components may include a size of light sensitive area that collects the light. The plurality of optical components 315 may enlarge the light collection area of the light detecting components based on the reflective material on the cone-shaped surface of the plurality of optical components 315.

The light emitting components may include additional optics through the use of the reflectors that direct the emitted beam towards the surface of the skin to enhance the light coupling and simultaneously block internal stray light. For example, the light may be directed towards the surface of the skin and deeper into the skin while less light may be scattered across the surface of the skin or into the sensor structure.

The reflective material of the plurality of optical components 315 may reflect an increased amount of light, increase signal quality, and enhance the output of the light emitting components to the surface of the skin. In such cases, the signals from the light detecting components are collected from a larger surface area around the dome structure. In some cases, the signal may increase 130% with infrared (IR) light. The stray light may be blocked, thereby causing a signal quality improvement. The reflective material (e.g., mirror coatings) may be positioned between the material of the plurality of optical components 315 and an epoxy material during the molding process.

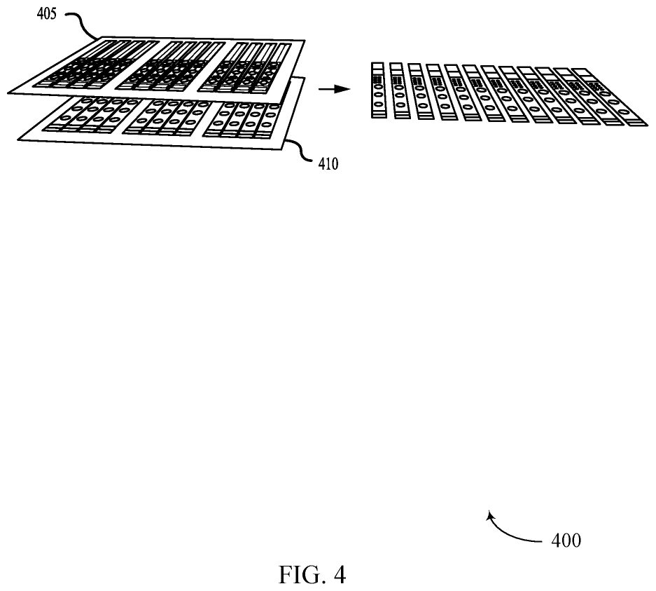

FIG. 4 shows an example of a manufacturing system 400 that supports optical components and assembly techniques in accordance with aspects of the present disclosure. The manufacturing system 400 may implement, or be implemented by, the system 100, the system 200, and the manufacturing system 300. In particular, the manufacturing system 400 illustrates a manufacturing process to manufacture a panel of a plurality of array optics assemblies, as described with reference to FIGS. 1 through 3.

The manufacturing system 400 may include a panel of a plurality of alignment substrates 405 and a panel of a plurality of optoelectronic components 410. The manufacturing system 400 may perform the manufacturing processes as described with reference to FIGS. 3A and 3B. Additionally, the manufacturing system 400 may align the panel of a plurality of alignment substrates 405 onto the panel of the plurality of optoelectronic components 410.

In such cases, a high throughput may be achieved with a panel level assembly such that multiple optical components deposited on the alignment substrates may be positioned on and aligned with the plurality of optoelectronic components as opposed to aligning each individual optical component on each individual optoelectronic component separately. For example, as described with reference to manufacturing system 400, sixty optical components may be adhered to twelve flexible PCBs that include the plurality of optoelectronic components. In such cases, manufacturing the panel of the plurality of array optics assemblies may reduce an amount of time to manufacture the array optics assemblies, thereby reducing a cost associated with the manufacturing while maintaining an accuracy of optical component placement.

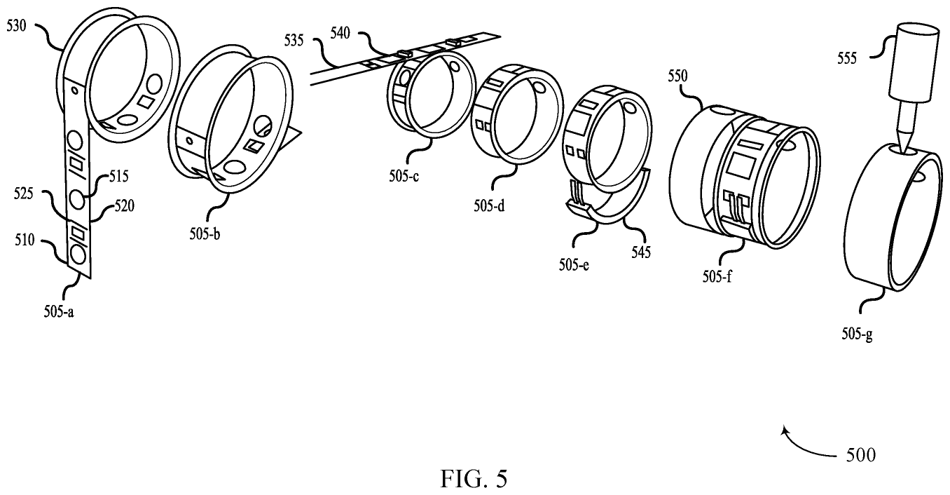

FIG. 5 shows an example of a manufacturing system 500 that supports optical components and assembly techniques in accordance with aspects of the present disclosure. The manufacturing system 500 may implement, or be implemented by, the system 100, the system 200, the manufacturing system 300, and the manufacturing system 400. In particular, the manufacturing system 500 illustrates a manufacturing process to manufacture a wearable ring device, as described with reference to FIGS. 1 through 4.

At processing step 505-a, the alignment substrate 510 may be positioned relative to an inner ring inlet 530. The alignment substrate 510 may include a plurality of optical components 515, an adhesive material 520, one or more light blocking components 525, or a combination thereof. For example, in addition to optics, the alignment substrate 510 may include features such as mechanical spacers and stray light blockers (e.g., light blocking components 525). The alignment substrate 510 including the plurality of optical components 515 may be manufactured according to the manufacturing process as described with reference to FIG. 3A.

The manufacturing system 500 may assemble the alignment substrate 510 onto an inner circumference of an inner ring inlet 530 (e.g., inner ring housing member) of a wearable ring device prior to adhering the alignment substrate 510 to the plurality of optoelectronic components 540. The inner ring inlet 530 may include a metallic material configured to block stray light. The manufacturing system 500 may position the alignment substrate 510 adjacent to the inner ring inlet 530 to integrate the optics (e.g., the plurality of optical components 515) into the metallic inner ring inlet 530 before adding the plurality of optical components 515 to the flexible PCB 535. The manufacturing process as described with reference to FIG. 5 may result in a reduction of cost and faster assembly due to the elimination of the molding process as described with reference to FIG. 3A.

At processing step 505-b, the manufacturing system 500 may adhere the alignment substrate 510 to the inner ring inlet 530. The alignment substrate 510 may include the adhesive material 520 such that the adhesive material 520 of the alignment substrate 510 is adhered to the inner ring inlet 530. For example, the plurality of optical components 515 may be attached, via the adhesive material 520, to the inner ring inlet 530. The manufacturing system 500 may wrap the alignment substrate 510 around the inner ring inlet 530. The adhesive material 520 may be on a first side of the alignment substrate 510 that contacts the inner ring inlet 530, a second side of the alignment substrate 510, or both.

The manufacturing system 500 may create a seal between the alignment substrate 510 and the inner ring inlet 530. For example, the inner ring inlet 530 may be integrated into the alignment substrate 510 such that the interface between the inner ring inlet 530 and the alignment substrate 510 is removed and the structure at processing step 505-b is sealed.

At processing step 505-c, the manufacturing system 500 may position the flexible PCB 535 adjacent to the alignment substrate 510 that circumscribes the inner ring inlet 530. The flexible PCB 535 may include the plurality of optoelectronic components 540. In some cases, the flexible PCB 535 may include an adhesive material on a first side that contacts the plurality of optoelectronic components 540, on a second side opposite the first side, or both. For example, the adhesive material of the flexible PCB 535 may contact the second side of the alignment substrate 510.

At processing step 505-d, the manufacturing system 500 may adhere the flexible PCB 535 to the alignment substrate 510. The alignment substrate 510 may include the adhesive material 520 such that the adhesive material 520 of the alignment substrate 510 is adhered to the flexible PCB 535. In such cases, the flexible PCB 535 may be attached to the alignment substrate 510. The manufacturing system 500 may wrap the flexible PCB 535 around the alignment substrate 510. The manufacturing system 500 may create a seal between the alignment substrate 510 and the flexible PCB 535. For example, the flexible PCB 535 may be integrated into the alignment substrate 310 such that the interface between the flexible PCB 535 and the alignment substrate 510 is removed and the structure at processing step 505-d is sealed.

In some cases, at processing step 505-e, a power source 545 (e.g., battery) may be soldered onto the flexible PCB 535. At processing step 505-f, an outer cover 550 may be assembled around the flexible PCB 535. At processing step 505-g, the manufacturing system 500 may inject a fillable material 555 through an opening in a wearable ring device such that the fillable material 555 contacts the flexible PCB 535. The fillable material may be an example of an epoxy material. In such cases, the epoxy material may include an opaque (e.g., black) material.

The manufacturing system 500 may fill the space between the inner ring inlet 530 and the outer cover 550 with the fillable material 555. In some cases, the fillable material 555 may fill the space between the plurality of optical components 515 such that stray light may be blocked effectively. The fillable material 555 may be an example of a layer of material that includes optical properties that propagate the light from the set of light emitting components to the set of light detecting components. In some cases, the fillable material 555 in combination with a reflective material of the plurality of optical components 515 may improve the battery life and increase the signal quality of the PPG signal.

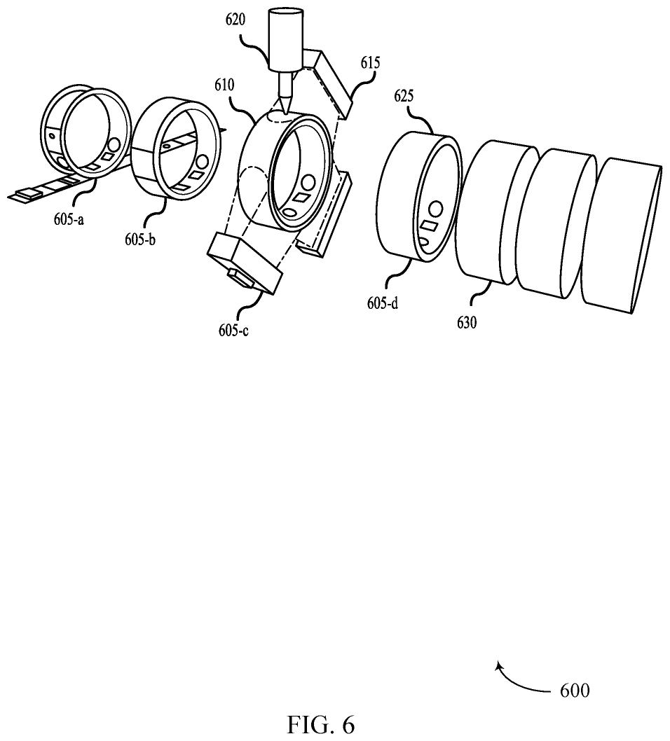

FIG. 6 shows an example of a manufacturing system 600 that supports optical components and assembly techniques in accordance with aspects of the present disclosure. The manufacturing system 600 may implement, or be implemented by, the system 100, the system 200, the manufacturing system 300, the manufacturing system 400, and the manufacturing system 500. In particular, the manufacturing system 600 illustrates a manufacturing process to manufacture a wearable ring device, as described with reference to FIGS. 1 through 5.

Processing steps 605-a and 605-b may be an example of processing steps 505-c through 505-e as described with reference to FIG. 5. In some cases, if the alignment substrate includes a double sided adhesive material (e.g., the adhesive material is included on the first side and the second side of the alignment substrate), the array optics assembly may be manufactured without epoxy molding. In such cases, the adhesive material may seal off the optical apertures of inner ring inlet.

At processing step 605-c, the manufacturing system 600 may coat the outer housing of the assembled electronics 610 with a material 620. The material 620 may be an example of an epoxy material or a UV curable glue. In such cases, the wearable ring device may be manufactured without a ring cover. The manufacturing system 600 may apply a light source 615 to the assembled electronics 610 to create a seal between the material 620 and the assembled electronics 610. In some examples, the manufacturing system 600 may apply UV light to the UV curable glue (e.g., material 620).

At processing step 605-d the manufacturing system 600 may seal an inner core 625 of the wearable ring device with UV curable glue, for example. The assembled electronics 610 may include a temporary mold that is transparent. The assembled electronics 610 may be UV cured through the transparent mold. In such cases, the manufacturing system 600 may enable the inner core 625 with sensor functionality to be swappable with different ring covers 630 of different colors, styles, materials, or a combination thereof. The appearance of the wearable ring device may vary based on the ring cover 630 while maintaining a consistent functionality. In some cases, the epoxy sealing may be used without the ring cover 630.

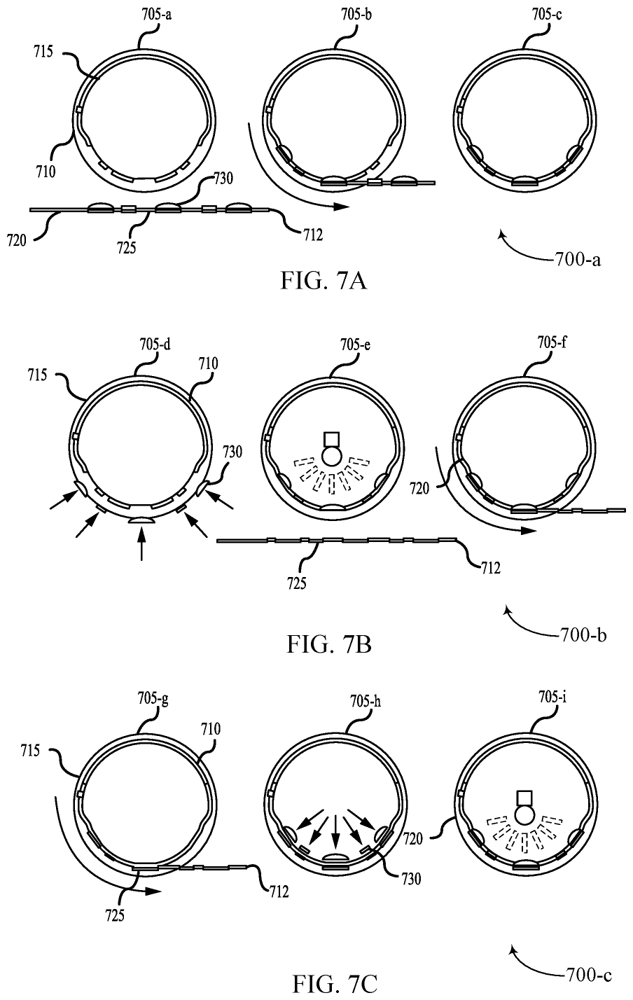

FIG. 7 shows examples of manufacturing systems 700 that support optical components and assembly techniques in accordance with aspects of the present disclosure. The manufacturing system 700 may implement, or be implemented by, the system 100, the system 200, the manufacturing system 500, and the manufacturing system 600. In particular, the manufacturing system 700 illustrates a manufacturing process to manufacture a wearable ring device, as described with reference to FIGS. 1, 2, 5, and 6.

Manufacturing system 700-a may illustrate a first type of manufacturing process to manufacture the wearable ring device sub-assembly 710. At processing step 705-a, a plurality of optical components 720 may be positioned relative to an inner ring inlet 715 of the wearable ring device sub-assembly 710. The plurality of optical components 720 be adhered to a substrate 712. The substrate 712 including the plurality of optical components 720 may be manufactured according to the manufacturing process as described with reference to FIG. 3A. The plurality of optical components 720 may include at least a first type of optical components 725 and a second type of optical components 730. The first type of optical components 725 are aligned with the second type of optical components 730.

The optical features configured for skin contact may be an example of the second type of optical components 730, and the optical features configured for modifying LED emission and/or photodetector field of view may be an example of the first type of optical components 725. For example, the first type of optical components 725 may be configured to manipulate a light emission direction or a light detection direction, and the second type of optical components 730 may be configured to at least partially seal the plurality of apertures. The first type of optical components 725 and the second type of optical components 730 may be located on a same substrate 712 (e.g., foil).

The manufacturing system 700-a may assemble the plurality of optical components 720 onto an inner circumference of an inner ring inlet 715 (e.g., inner ring housing member) of a wearable ring device sub-assembly 710 prior to assembling a plurality of optoelectronic components to the plurality of optical components 720. The inner ring inlet 715 may include a metallic material configured to block stray light. The manufacturing system 700-a may position the plurality of optical components 720 adjacent to the inner ring inlet 715 to integrate the optics (e.g., the plurality of optical components 720) into the metallic inner ring inlet 715 before adding the flexible PCB (e.g., including plurality of optoelectronic components) to the plurality of optical components 720. The manufacturing process may result in a reduction of cost and faster assembly due to the elimination of the molding process as described with reference to FIG. 3A.

At processing step 705-b, the manufacturing system 700-a may adhere the plurality of optical components 720 to the inner ring inlet 715. For example, the substrate 712 with the plurality of optical components 720 may be placed to a positioning feature on the inner ring inlet 715. The positioning feature may be an example of mechanical features such as a hook, a clip, a tap, an adhesive material, and the like. The substrate 712 that includes the plurality of optical components 720 may include an adhesive material such that the adhesive material of the substrate 712 is adhered to the inner ring inlet 715. For example, the plurality of optical components 720 may be attached, via the adhesive material, to the inner ring inlet 715 by applying pressure to the adhesive material.

The manufacturing system 700-a may wrap the substrate 712 including the plurality of optical components 720 around the inner ring inlet 715. The adhesive material may be on a first side of the substrate 712 that contacts the inner ring inlet 715, a second side of the substrate 712 opposite of the first side, or both. As the substrate 712 is wrapped around the inner ring inlet 715, the plurality of optical components 720 may be attached to the inner ring inlet 715 over the inlet apertures (e.g., filling the optical apertures). In such cases, the plurality of optical components 720 may be aligned with a plurality of apertures of the inner ring inlet 715. The substrate 712 may be wrapped around the inner ring inlet 715 in a single process.

At processing step 705-c, the manufacturing system 700-a may create a seal between the substrate 712 including the plurality of optical components 720 and the inner ring inlet 715. For example, the inner ring inlet 715 may be integrated into the substrate 712 such that the interface between the inner ring inlet 715 and the substrate 712 (e.g., the plurality of optical components 720) is removed and the structure at processing step 705-c is sealed. In some cases, the seal may be between the plurality of optical components 720 and the plurality of apertures (e.g., optical apertures) of the inner ring inlet 715. In such cases, the optical apertures may be sealed and the plurality of optical components 720 may be fixed to the inner ring inlet 715.

After processing step 705-c, the manufacturing system 700-a may position the flexible PCB adjacent to the substrate 712 including the plurality of optical components 720 that circumscribes the inner ring inlet 715. The flexible PCB may include the plurality of optoelectronic components. The plurality of optoelectronic components may include a set of light emitting components and a set of light detecting components. In some cases, the flexible PCB may include an adhesive material on a first side that contacts the plurality of optoelectronic components, on a second side opposite the first side, or both. For example, the adhesive material of the flexible PCB may contact the second side of the substrate 712.

After processing step 705-c, the manufacturing system 700-a may adhere the flexible PCB to the substrate 712 including the plurality of optical components 720. The adhesive material of the substrate 712 may be adhered to the flexible PCB. In such cases, the flexible PCB may be attached to the substrate 712 including the plurality of optical components 720. The manufacturing system 700-a may wrap the flexible PCB around the substrate 712 including the plurality of optical components 720. The first type of optical components 725 may be positioned between the plurality of optoelectronic components and the second type of optical components 730. The manufacturing system 700-a may create a seal between the plurality of optical components 720 and the flexible PCB. For example, the flexible PCB may be integrated into the substrate 712 such that the interface between the flexible PCB and the substrate 712 is removed and the structure is sealed.

In some cases, a power source (e.g., battery) may be soldered onto the flexible PCB. An outer cover may be assembled around the flexible PCB. The manufacturing system 700-a may then inject a fillable material through an opening in a wearable ring device sub-assembly 710 such that the fillable material contacts the flexible PCB. The fillable material may be an example of an epoxy material. In such cases, the epoxy material may include an opaque (e.g., black) material.

The manufacturing system 700-a may fill the space between the inner ring inlet 715 and the cover with the fillable material. In some cases, the fillable material may fill the space between the plurality of optical components 720 such that stray light may be blocked effectively. The fillable material may be an example of a layer of material that includes optical properties that prevent propagation of the light from the set of light emitting components to the set of light detecting components. In some cases, the fillable material in combination with a reflective material of the plurality of optical components 720 may improve the battery life and increase the signal quality of the PPG signal.

In some cases, the inner optical apertures of the inner ring inlet 715 may be sealed with the optics such that the wearable ring device sub-assembly 710 may be resistant to water, moisture, dirt, and the like. The airspace between the inner ring inlet 715 and the outer cover of the wearable ring device sub-assembly 710 may be filled by epoxy molding after the assembly if the optical interfaces between the optoelectronic components and the plurality of optical components 720 are protected such that the material does not fill the airgap used for optical functions.

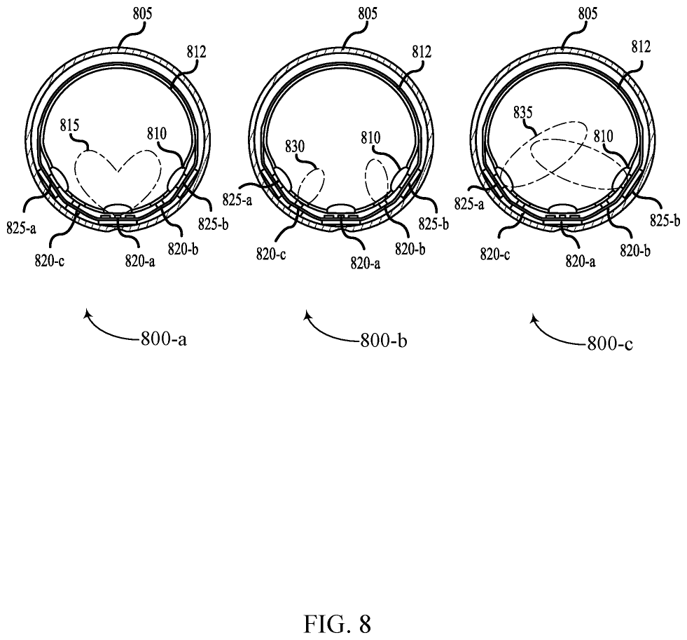

The manufacturing system 700-a may assemble the optical arrays (e.g., including the plurality of optical components 720) as stickers to the inner ring inlet 715. The ring PPG sensor (e.g., optoelectronic components) of the wearable ring device sub-assembly 710 may include optical features on the side of the inner ring inlet 715 with optical functions (e.g., the inner circumference). For example, the optical functions may include creating a contact between the optical material covering optoelectronic components and the skin surface for light injection and extraction from the tissue. The LED emission pattern and photodetector field of view may be modified to optimize PPG measurement, as described herein with reference to FIG. 8.

Manufacturing system 700-b may illustrate a second type of manufacturing process to manufacture the wearable ring device sub-assembly 710, alternative to the first type of manufacturing process. At processing step 705-d, a second type of optical components 730 may be positioned relative to an inner ring inlet 715 of the wearable ring device sub-assembly 710. In such cases, the separate skin-touching components (e.g., the second type of optical components 730) may be assembled to the inner ring inlet 715 as a first step. The second type of optical components 730 that contact the skin directly may be assembled to the inner ring inlet 715 as separate pieces. The manufacturing system 700-b may position the second type of optical components 730 adjacent to the inner ring inlet 715 to integrate the optics (e.g., the second type of optical components 730) into the metallic inner ring inlet 715 before adding the first type of optical components to the inner ring inlet 715. The manufacturing process may result in a reduction of cost and faster assembly due to the elimination of the molding process as described with reference to FIG. 3A.

At processing step 705-c, the manufacturing system 700-b may attach the first type of optical components 730 to the inner ring inlet 715 by UV curing. In such cases, at processing step 705-e, the manufacturing system 700-b may adhere the second type of optical components 730 to the inner ring inlet 715. In some examples, the manufacturing system 700-b may apply a light source to the second type of optical components 730 to create a seal between the second type of optical components 730 and the inner ring inlet 715 after adhering the second type of optical components 730 to the inner ring inlet 715. For example, the manufacturing system 700-b may apply UV light to the second type of optical components 730. In such cases, the second type of optical components 730 may be integrated onto the inner ring inlet 715 such that the interface between the second type of optical components 730 and the inner ring inlet 715 is removed and the structure (e.g., including the second type of optical components 730 and the inner ring inlet 715) is sealed. For example, the manufacturing system 700-b may create a seal between the plurality of optical components 720 and the plurality of apertures of the inner ring inlet 715.

At processing step 705-e, the first type of optical components 725 may be adhered to a substrate 712. The substrate 712 may be manufactured according to the manufacturing process as described with reference to FIG. 3A. The first type of optical components 725 may be positioned relative to an inner ring inlet 715 of the wearable ring device sub-assembly 710. The manufacturing system 700-b may position the first type of optical components 725 adjacent to the inner ring inlet 715 to integrate the optics (e.g., the first type of optical components 725) into the metallic inner ring inlet 715 before adding the flexible PCB including plurality of optoelectronic components to the inner ring inlet 715. In such cases, the optics configured for modifying LED emission and/or photodetector field of view (e.g., the first type of optical components 725) are located on the substrate 712 (e.g., foil) while the second type of optical components 730 are positioned on the inner ring inlet 715 separate from the substrate 712.

At processing step 705-f, the manufacturing system 700-b may assemble the first type of optical components 725 onto an inner circumference of an inner ring inlet 715 (e.g., inner ring housing member) of a wearable ring device sub-assembly 710 prior to assembling a plurality of optoelectronic components to the plurality of optical components 720. The manufacturing system 700-b may adhere the substrate 712 including the first type of optical components 725 to the inner ring inlet 715. The substrate 712 that includes the first type of optical components 725 may include an adhesive material such that the adhesive material of the substrate 712 is adhered to the inner ring inlet 715. For example, the first type of optical components 725 may be attached, via the adhesive material, to the inner ring inlet 715 by applying pressure to the adhesive material. The adhesive material may be on a first side of the substrate that contacts the inner ring inlet 715, a second side of the substrate 712 opposite of the first side, or both.

The manufacturing system 700-b may wrap the substrate 712 including the first type of optical components 725 around the inner ring inlet 715 that includes the second type of optical components 725. The light-manipulating optical components (e.g., the first type of optical components 725) may be wrapped around the inner ring inlet 715 with filled apertures. In such cases, the first type of optical components 725 are aligned with the second type of optical components 730.

At processing step 705-f, the manufacturing system 700-b may create a seal between the plurality of optical components 720 and the inner ring inlet 715. For example, the inner ring inlet 715 may be integrated into the substrate 712 such that the interface between the inner ring inlet 715 and the plurality of optical components 720 is removed and the structure at processing step 705-f is sealed. In such cases, the manufacturing system 700 may create a seal between the plurality of optical components 720 and the plurality of apertures of the inner ring inlet 715.

After processing step 705-f, the manufacturing system 700-b may position the flexible PCB adjacent to the plurality of optical components 720 that circumscribes the inner ring inlet 715. The flexible PCB may include the plurality of optoelectronic components and an adhesive material configured to attach the flexible PCB to the plurality of optical components 720. The manufacturing system 700-b may wrap the flexible PCB around the plurality of optical components 720. In some cases, the manufacturing system 700-b may create a seal between the plurality of optical components 720 and the flexible PCB. After processing step 705-f, the manufacturing system 700-b may perform the steps of attaching the flexible PCB, the battery, the cover, and/or adding the fillable material similar to the manufacturing system 700-a.

Manufacturing system 700-c may illustrate a third type of manufacturing process to manufacture the wearable ring device sub-assembly 710 as an alternative to the first type and second type of manufacturing process. At processing step 705-g, the first type of optical components 725 may be adhered to a substrate 712. The substrate 712 may be manufactured according to the manufacturing process as described with reference to FIG. 3A. The first type of optical components 725 may be positioned relative to an inner ring inlet 715 of the wearable ring device sub-assembly 710.

The manufacturing system 700-c may position the first type of optical components 725 adjacent to the inner ring inlet 715 to integrate the optics (e.g., the first type of optical components 725) into the metallic inner ring inlet 715 before adding the second type of optical components 730 (e.g., and the flexible PCB including plurality of optoelectronic components) to the inner ring inlet 715. In such cases, the optics configured for modifying LED emission and/or photodetector field of view (e.g., the first type of optical components 725) are located on the substrate 712 (e.g., foil) while the second type of optical components 730 are positioned on the inner ring inlet 715 separate from the substrate 712. The substrate 712 with the first type of optical components 725 (e.g., the light-manipulating optical components) may be assumed as a first step at processing step 705-g.

At processing step 705-g, the manufacturing system 700-c may assemble the first type of optical components 725 onto an inner circumference of an inner ring inlet 715 (e.g., inner ring housing member) of a wearable ring device sub-assembly 710 prior to assembling the second type of optical components 730 and the plurality of optoelectronic components. The manufacturing system 700-c may adhere the substrate 712 including the first type of optical components 725 to the inner ring inlet 715. The substrate 712 that includes the first type of optical components 725 may include an adhesive material such that the adhesive material of the substrate 712 is adhered to the inner ring inlet 715. For example, the first type of optical components 725 may be attached, via the adhesive material, to the inner ring inlet 715 by applying pressure to the adhesive material. The manufacturing system 700-c may wrap the substrate including the first type of optical components 725 around the inner ring inlet 715.

At processing step 705-g, the manufacturing system 700-c may create a seal between the first type of optical components 725 and the inner ring inlet 715. For example, the inner ring inlet 715 may be integrated into the substrate 712 such that the interface between the inner ring inlet 715 and the first type of optical components 725 is removed and the structure at processing step 705-g is sealed.

At processing step 705-h, a second type of optical components 730 may be positioned relative to an inner ring inlet 715 of the wearable ring device sub-assembly 710. The second type of optical components 730 that contact the skin directly may be assembled to the inner ring inlet 715 as separate pieces (e.g., each second type of optical components 730 may be attached consecutively to the inner ring inlet 715). The manufacturing system 700-c may position the second type of optical components 730 adjacent to the inner ring inlet 715 to integrate the optics (e.g., the second type of optical components 730) into the metallic inner ring inlet 715 before adding the plurality of optoelectronic components to the inner ring inlet 715 but after adding the first type of optical components 725 to the inner ring inlet 715. In such cases, the first type of optical components 725 are aligned with the second type of optical components 730. The manufacturing process may result in a reduction of cost and faster assembly due to the elimination of the molding process as described with reference to FIG. 3A.

At processing step 705-i, the manufacturing system 700-c may attach the plurality of optical components 720 (e.g., including the first type optical components 725 and the second type of optical components 730) to the inner ring inlet 715 by UV curing. In such cases, at processing step 705-i, the manufacturing system 700-c may adhere the plurality of optical components 720 to the inner ring inlet 715. In some examples, the manufacturing system 700-c may apply a light source to the plurality of optical components 720 to create a seal between the plurality of optical components 720 and the inner ring inlet 715 after adhering the second type of optical components 730 to the inner ring inlet 715. For example, the manufacturing system 700-c may apply UV light to the plurality of optical components 720. In such cases, the plurality of optical components 720 may be integrated onto the inner ring inlet 715 such that the interface between the plurality of optical components 720 and the inner ring inlet 715 is removed and the structure (e.g., including the plurality of optical components 720 and the inner ring inlet 715) is sealed.

After processing step 705-i, the manufacturing system 700-c may position the flexible PCB adjacent to the plurality of optical components 720 that circumscribes the inner ring inlet 715. After processing step 705-i, the manufacturing system 700-c may perform the steps of attaching the flexible PCB, the battery, the cover, and/or adding the fillable material similar to the manufacturing systems 700-a and 700-b.

FIG. 8 shows an example of wearable device diagrams 800 that supports optical components and assembly techniques in accordance with aspects of the present disclosure. The wearable device diagrams 800 may implement, or be implemented by, aspects of the system 100, system 200, the manufacturing system 300, the manufacturing system 400, the manufacturing system 500, the manufacturing system 600, and the manufacturing system 700. For example, wearable device diagrams 800 may illustrate examples of wearable devices 104 as described with reference to FIG. 1. Specifically, the wearable device diagrams 800 may illustrate an orientation of a wearable ring device on a user's finger. Although the wearable device is illustrated as a ring in FIG. 8, it may be any example of a wearable device (e.g., a watch, a necklace, and the like).

The wearable device 805 in wearable device diagram 800 may include an inner housing and an outer housing, which may be examples of an inner housing 205 and an outer housing 206 as described with reference to FIG. 2. In some cases, an outer opaque shell may be molded over an inner structure of the wearable device 805. Further, the wearable device 805 in the wearable device diagram 800 may include an electronic substrate 812, such as a printed wiring board (PWB) or PCB. The PWB may have both flexible and rigid sections. One or more sensors may be embedded in the electronic substrate 812. For example, the electronic substrate 812 may include one or more light sources 820 and detectors 825. The light sources 820 may be an example of LED lights may be a blue LED light, a yellow LED light, a green LED light, a red light, an IR light, or some other color LED light. In some cases, the light sources 820 may be an example of a laser diode (LD).

The wearable device 805 may include light source 820-a, which may emit light received by detector 825-a and/or detector 825-b. In this regard, the light source 820-a may support one or more optical paths through the tissue for physiological data measurements. For instance, the light source 820-a may support an optical path between the light source 820-a and the detector 825-a and another optical path between the light source 820-a and the detector 825-b. The wearable device 805 may include any number of light sources, detectors, and respective optical paths for physiological data measurements. In some cases, the light source 820-a may be a red and infrared LED, which may emit light that is scattered and absorbed by the tissue of a user of the wearable device 805.

Similarly, the wearable device 805 may include light source 820-b and light source 820-c. For example, the light source 820-b may emit light. The light source 820-b and the light source 820-c may be green LEDs, blue LEDs, or a combination thereof (e.g., one blue LED and one green LED). The light may be scattered and absorbed by the tissue of the user, and measured via the detectors 825-a and/or 825-b. As noted previously herein, each of the light sources 80-b and 820-c may support one or more optical paths via the respective detectors 825-a and 825-b. For instance, the light source 820-b may support an optical path between the light source 820-b and the detector 825-b and another optical path between the light source 820-b and the detector 825-a. The light source 820-c may support an optical path between the light source 820-c and the detector 825-b and another optical path between the light source 820-c and the detector 825-a.

The detectors 825-a and 825-b may be configured to measure light from the respective light sources 820 which is reflected by the tissue and/or transmitted through the tissue (e.g., reflective and/or transmissive measurements). In such cases, the light may be used for physiological data measurements associated with the user.

In some examples, the inner housing may include a dome structure over the one or more light sources 820, one or more detectors 825, or both. For example, the wearable device 805 may include dome structures over the light source 820-a, the detector 825-a, and the detector 825-b to improve contact with the tissue. In some other cases, there may be a window for the light source 820 to emit the light. For example, the light source 820-b and the light source 820-c may each have a window in the inner housing. An optical interface may form between the inner housing and the domes or the windows and the top layer of the tissue. The wearable device 805 may use the light propagation from the light sources 820 to the detectors 825 through the tissue and along the one or more optical paths for physiological measurements, such as PPG and SpO2 measurements. That is, the wearable device 805 may use the light from the light source 820-a, which may include red and infrared wavelengths, to measure SpO2 and the light from the light source 820-b or light source 820-c, which may include green wavelengths, to measure PPG.

In some examples, the wearable device 805 may be subjected to a force or an acceleration, causing an air gap between the surface of the tissue and one or more sensors at the wearable device 805. The air gap between the tissue and the optical components 810 may disturb the optical paths, as light may be coupled to the tissue through two interfaces (e.g., the interface between the optical components 810 and the air and the interface between the air and the tissue). Additionally, or alternatively, liquid or other contaminants may be trapped between the tissue and the optical components 810. The contaminants may dampen or absorb the optical signals. Further, the difference between refractive indexes and contaminant layer absorption spectra may determine how different signal paths/channels may be affected (e.g., causing increased variability in signal strength).

Domes on top of the light sources 820, such as on top of light source 820-a, may create steeper light incidence angles in the optical components 810 and the tissue or gap interface. The domes also protrude inside relatively elastic tissue, improving contact. However, they may not be sufficiently large to solve disturbance to the in-coupled signal. Thus, any changes in the amount of light coupled into the tissue may be compensated by controllers or drive electronics of the wearable device, causing additional losses in battery life as well as interruptions and inaccuracy to physiological measurements (e.g., PPG and SpO2 measurements). In some cases, SpO2 measurements may be affected by losses of in-coupled light and disturbance due to the oxygen saturation levels of blood being calculated as a ratio of two signals measured with two different wavelengths from light sources 820. As the two light propagation paths are both spatially and angularly different from each other, changes to either of the signal paths may cause measurement inaccuracy.

The wearable device 805 may include a plurality of optical components 810. In some cases, the optical components 810 may interface with the inner housing and the surface of tissue. The optical components 810 may be disposed on the surface of the housing (e.g., inner housing). In some examples, the optical components 810 may be molded from a material (e.g., epoxy, plastic, metal, and the like) that is capable of transmitting or reflecting light. That is, the optical components 810 may have optical properties that allow the optical components 810 to propagate light from the light sources 820 within the inner housing.

The optical components 810 may be an example of a lens, a microprism, a reflector, a diffractive structure, a metamaterial lens, a metallic material configured for optics that includes an optical functionality, a prism, a micro lens array, an angular filter, or a combination thereof. The optical components 810 may be configured to alter the light pattern of the light emitted from the light sources 820 and the light detected by detectors 825. For example, the first type of optical components may be configured to manipulate a light emission direction or a light detection direction. The second type of optical components may be configured to at least partially seal the plurality of apertures. With reference to wearable device diagram 800-a, the light source 820-a may include a light emission pattern 815. The light emission pattern 815 may include a beam width (e.g., a light emission size), a beam shape (e.g., a light emission shape), a beam direction (e.g., light emission direction), a beam angle (e.g., a light emission tilt angle), or a combination thereof.

Without the optical components 810, the light emission pattern 815 may include a uniform structure of even distribution that is direct towards the center of the tissue which may be inefficient for physiological data measurements. However, with the use of the optical components 810, the light emission pattern 815 may be directed into at least two directions (e.g., two halves) to form a heart-shaped light emission pattern 815 pattern. The light may be directed towards the sides of the tissue and towards the detectors 825 rather than the center of the tissue (e.g., middle of the finger). By modifying the light emission pattern 815, the signal quality may increase and the overall efficiency of the wearable device 805 may increase. The light emission pattern 815 may be directed into a plurality of direction to form a plurality of shapes.

With reference to wearable device diagram 800-b, the light source 820-c and light source 820-b may include light emission pattern 830. The light emission patterns 830 may each include a beam width, a beam shape, a beam direction, a beam angle (e.g., tilt), or a combination thereof. In some cases, if the light emission pattern 830 is too close to the detectors 825, the amount of stray light may increase. When the light sources 820 lose contact with the tissue (e.g., due to gaps that may be caused by motion artifacts), more stray light may be produced. For example, the light sources 820-b and 820-c may produce more stray light than the light source 820-a because the light sources 820-b and 820-c are closer to the detectors 825 than the light source 820-a. Further, because the light sources 820-b and 820-c are closer to the edges of the domes covering the detectors 825, the light sources 820-b and 820-c may lose contact with the tissue more dynamically than light source 820-a since the domes may stretch the tissue.

Additionally, or alternatively, the light sources 820-b and 820-c may produce more stray light than the light source 820-a because neither the light source 820-b nor the light source 820-c are covered by a dome. Because the light source 820-a is covered by a dome, the light source 820-a may emit light with better light incident angles for coupling of the emitted light into the tissue (e.g., in-coupling) because the light incident angles are less steep. However, because the light sources 820-b and 820-c are uncovered by domes, the light sources 820-b and 820-c may emit light with steeper light incident angles, resulting in more coupling of the emitted light to the epoxy of the optical components 810 and less in-coupling. In such cases, the lack of domes over the light sources 820-b and 820-c may also contribute to the emission of a greater amount of stray light from the light sources 820-b and 820-c than from the light source 820-a.

By including the optical components 810 in the wearable device 805 (e.g., over the light source 820-c and light source 820-b), the light emission pattern 830 may be modified to direct light deeper into the tissue rather than directed towards the detectors 825. In such cases, less stray light propagating both inside the ring structure and in the superficial layers of the skin may be emitted, and the light received by the detectors 825 may be from light coming from deeper structures within the tissue. The tilt direction of the light emission pattern 830 may be modified towards the center of the tissue rather than towards the detectors 825 in order to contribute additional light distributions to the detectors 825.

With reference to wearable device diagram 800-c, the detector 825-a and detector 825-a may include respective field of views 835. The field of views 835 may be modified based on including the optical components 810 on the detectors 825. The optical components 810 may enable the field of view 835 of the detectors 825 to focus the light detection in a specific direction or even-tilted direction rather than distributing the field of view 835 to a wide range of angles. In such cases, the optical components 810 may enable the field of view 835 to be modified, which is the integrated area of the tissue that is illuminated by the light emitted from the light sources 820 but also detected by the detectors 825. The field of view 835 may include a set of characteristics. For example, the field of view 835 may include a light detection direction, a light direction tilt angle, a light detection size, a light detection shape, or a combination thereof.

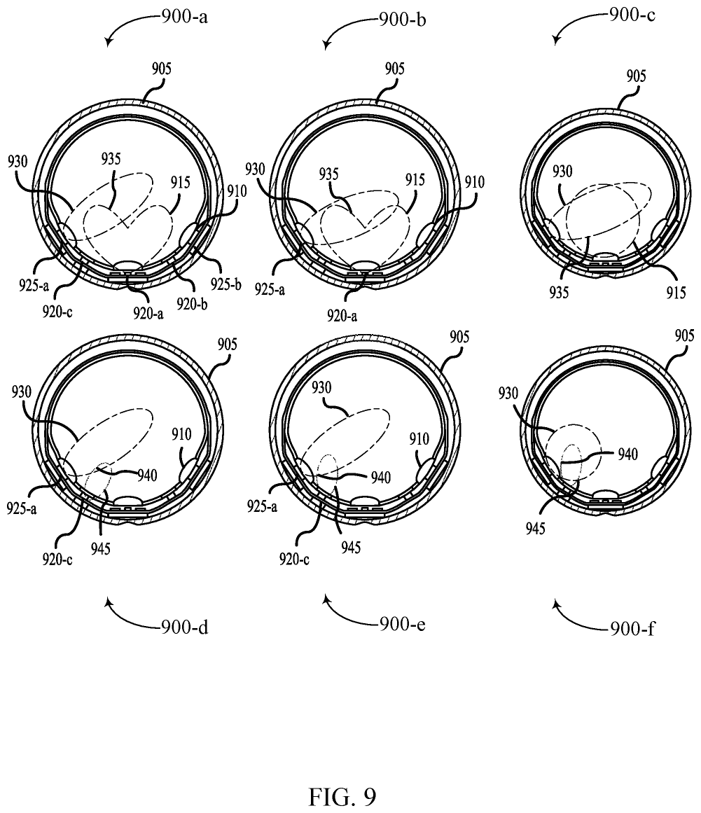

FIG. 9 shows an example of wearable device diagrams 900 that supports optical components and assembly techniques in accordance with aspects of the present disclosure. The wearable device diagrams 900 may implement, or be implemented by, aspects of the system 100, system 200, the manufacturing system 300, the manufacturing system 400, the manufacturing system 500, the manufacturing system 600, the manufacturing system 700, and wearable device diagrams 800. For example, wearable device diagrams 900 may illustrate examples of wearable devices as described with reference to FIGS. 1 and 8. Specifically, the wearable device diagrams 900 may illustrate an orientation of a wearable ring device on a user's finger. Although the wearable device is illustrated as a ring in FIG. 9, it may be any example of a wearable device (e.g., a watch, a necklace, and the like).

The wearable device 905 may include the plurality of optical components 910 as described with reference to FIGS. 7 and 8. The optical components 910 may be configured to alter the light pattern of the light emitted from the light sources 920 and the light detected by detectors 925, as described with reference to FIG. 8.

With reference to wearable device diagram 900-a, the optical components 910 disposed over at least the light source 920-a may be used to direct the light emission pattern 915, as described with reference to wearable device diagram 800-a. For example, the light emission pattern 915 may be directed into at least two directions to form a heart-shaped light emission pattern 915. The optical components 910 disposed over the detector 925-a may modify the field of view 930 of the detector 925-a to focus in a specific direction. In such cases, the combination of the optical components 910 on the light source 920-a and the detector 925-a may be used together to modify both the light emission pattern 915 and the field of view 930 to form an overlapping portion 935 of the light emission pattern 915 and the field of view 930.

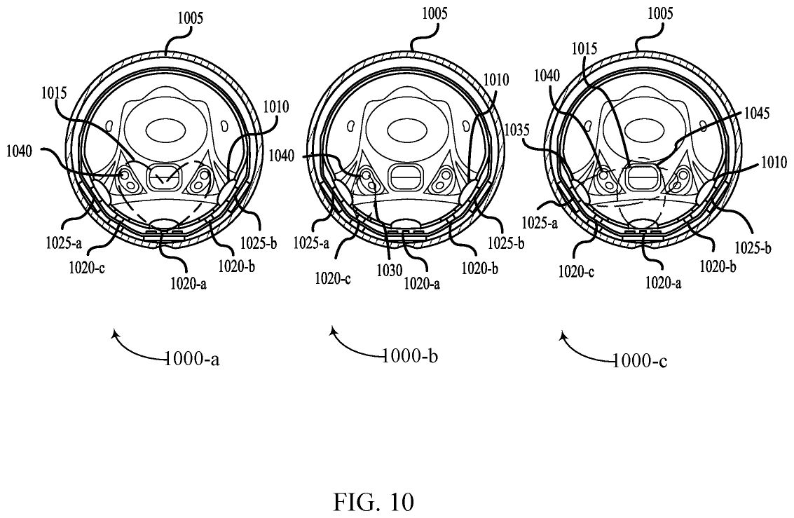

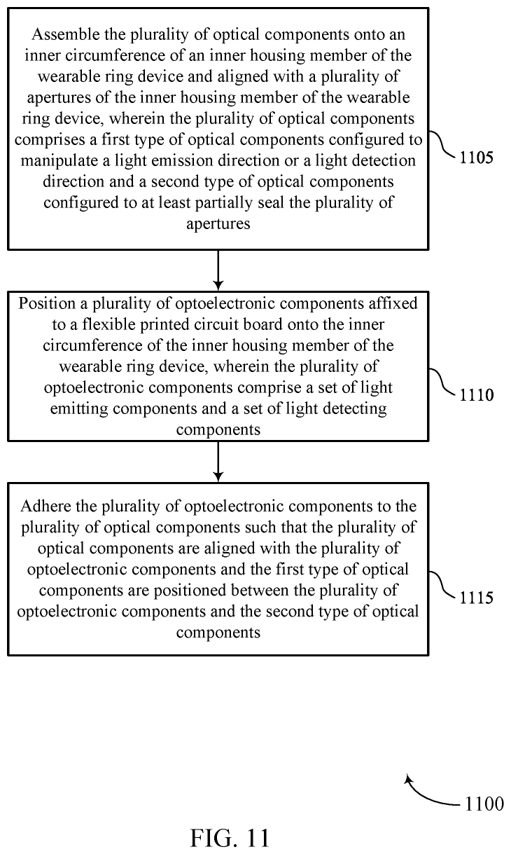

With reference to wearable device diagram 900-b, the tilt of the angle for the field of view 930 may be updated to form a greater overlapping portion 935 as compared to the overlapping portion 935 of the wearable device diagram 900-a. In such cases, the optical component 910 disposed over the detector 925-a may be a different material, different thickness, and the like to alter the tilt of the angle for the field of view 930 in the wearable device diagram 900-b. The overlapping portion 935 may target a specific location in the human physiology (e.g., the arteries in the digit of the user, as further described herein with reference to FIG. 10). In some cases, the optical components 910 may tilt the field of view 930 such that the overlapping portion 935 may increase, thereby increasing an area of impact (e.g., an area that the light propagates within the tissue).

In some cases, the distance between the detector 925 and the overlapping portion 935 may affect the amount of signal received at the detectors 925. For example, as the distance between the detector 925 and the overlapping portion 935 increases, the amount of signal received at the detectors 925 may decrease. For example, if the intersection of the field of view 930 and light emission pattern 915 is closer to the detector 925 (e.g., within a threshold distance), an amount of signal received at the detector 925 may increase. That is, a distance between the overlapping portion 935 and the detector 925 may determine an amount of signal received at the detector 925.

As the distance between the detector 925-a and the overlapping portion 935 decreases, the quantity, quality, and/or efficiency of the signal may increase, thereby reducing the overall power consumption of the battery. The distance may be an example of a distance between the detector 925 and a center of the overlapping portion 935, an intersection point between the field of view 930 and the light emission pattern 915, and the like. In some cases, a location of the overlapping portion 935 may be adjusted to target physiology within the user, as described further with reference to FIG. 10.

With reference to wearable device diagram 900-d, the optical components 910 disposed over at least the light source 920-c may be used to direct the light emission pattern 945, as described with reference to wearable device diagram 800-b. The optical components 910 disposed over the detector 925-a may modify the field of view 930 of the detector 925-a to focus in a specific direction. In such cases, the combination of the optical components 910 on the light source 920-c and the detector 925-a may be used together to modify both the light emission pattern 945 and the field of view 930 to form an overlapping portion 940 of the light emission pattern 945 and the field of view 930.

With reference to wearable device diagram 900-e, the tilt of the angle for the field of view 930 and the tilt of the angle for the light emission pattern 945 may be updated to form a greater overlapping portion 940 as compared to the overlapping portion 940 of the wearable device diagram 900-d. In such cases, the optical components 910 disposed over the detector 925-a and the light source 920-c may be a different material, different thickness, and the like to alter the tilt of the angle for the field of view 930 in the wearable device diagram 900-d.

In such cases, the overlapping portion 940 may target a specific location in the human physiology (e.g., an artery in the digit of the user, as further described herein with reference to FIG. 10). The optical components 910 may tilt the field of view 930 such that the overlapping portion 940 may increase, thereby increasing an area of impact (e.g., an area that the light propagates within the tissue). In some cases, the greater the distance between the detector 925 and the overlapping portion 940, the less signal received at the detectors 925. For example, if the intersection of the field of view 930 and light emission pattern 945 is closer to the detector 925 (e.g., within a threshold distance), an amount of signal received at the detector 925 may increase. That is, a distance between the overlapping portion 940 and the detector 925 may determine an amount of signal received at the detector 925. As the distance between the detector 925-a and the overlapping portion 940 decreases, the quantity, quality, and/or efficiency of the signal may increase, thereby reducing the overall power consumption of the battery. In some cases, a location of the overlapping portion 940 may be adjusted to target physiology within the user, as described further with reference to FIG. 10.

In some cases, a size of the wearable device 905 may affect the field of view 930, the light emission pattern 915, the light emission pattern 945, an overlapping portion 935, or a combination thereof. That is, a change in ring size may change a distance between the light sources 920 and the detectors 925. As described with reference to wearable device diagram 900-c, a decreased diameter of the wearable device 905 may adjust the field of view 930, the light emission pattern 915, an overlapping portion 935, or a combination thereof. For example, the field of view 930 and/or the light emission pattern 915 may increase as the diameter of the wearable device 905 decreases. In such cases, the size of the field of view 930, the size of the light emission pattern 915, or both may increase relative to the diameter of the wearable device 905. The overlapping portion 935 may increase as the diameter of the wearable device 905 decreases. The amount of signal received at the detectors 925 may thereby increase as the distance between the overlapping portion 935 and the detectors 925 increases.

Similarly, as described with reference to wearable device diagram 900-f, as the diameter of the wearable device 905 decreases, the field of view 930, the light emission pattern 945, an overlapping portion 940, or a combination thereof may be adjusted. For example, the field of view 930, the light emission pattern 945, the overlapping portion 940, or a combination thereof may increase as the diameter of the wearable device 905 decreases.

FIG. 10 shows an example of wearable device diagrams 1000 that supports optical components and assembly techniques in accordance with aspects of the present disclosure. The wearable device diagrams 1000 may implement, or be implemented by, aspects of the system 100, system 200, the manufacturing system 300, the manufacturing system 400, the manufacturing system 500, the manufacturing system 600, the manufacturing system 700, wearable device diagrams 800, and wearable device diagrams 900. For example, wearable device diagrams 1000 may illustrate examples of wearable devices as described with reference to FIGS. 1, 8, and 9. Specifically, the wearable device diagrams 1000 may illustrate an orientation of a wearable ring device on a user's finger. Although the wearable device is illustrated as a ring in FIG. 10, it may be any example of a wearable device (e.g., a watch, a necklace, and the like).

The wearable device 1005 may include a plurality of optical components 1010. In some examples, the optical components 1010 may be molded from a material (e.g., epoxy, plastic, metal, and the like) that is capable of transmitting light. That is, the optical components 1010 may have optical properties that allow the optical components 1010 to propagate light from the light sources 1020 within the inner housing. The optical components 1010 may be an example of passive optical components. For example, the optical components 1010 may be an example of a microprism, a diffracted structure, a metamaterial lens (e.g., component), a metal material configured for optics that includes an optical functionality, or a combination thereof. For example, the optical components 1010 may be an example of liquid crystal-based components (e.g., gratings) that may be adjusted electrically to modify the optical function, beam shifters, steering mirrors, focus tunable lenses, or a combination thereof. In such cases, the ring PPG sensor measurement may be switched from a HR measurement to SpO2 measurement for the optimized configuration. The optical components 1010 may be configured to alter the light pattern of the light emitted from the light sources 1020 and the light detected by detectors 1025, as described with reference to FIGS. 8 and 9.

With reference to wearable device diagram 1000-a, the optical components 810 disposed over at least the light source 1020-a may be utilized to target the light emission pattern 1015 to a specific point in the physiology of the user. For example, the light emission pattern 1015 may be directed to target the arteries 1040 on either side of the user's finger by utilizing the optical components 1010. That is, the heart-shaped light distribution pattern of light emission pattern 1015 may direct light to the arteries 1040 rather than the center of the tissue (e.g., thereby bypassing the arteries 1040).

With reference to wearable device diagram 1000-b, the optical components 810 disposed at least over light source 1020-c may be utilized to target the light emission 1030 to a specific point in the physiology of the user (e.g., the arteries 1040). Directing the light emitted from lights sources 1020-c and 1020-b towards the arteries 1040 may result in a more accurate physiological data signal as opposed to directing light at the center of the tissue and away from the arteries 1040. With reference to wearable device diagram 1000-c, the optical components 1010 disposed over at least the detector 1025-a and the light source 1020-a may be utilized to modify the field of view 1035 of the detector 1025-a and the light emission pattern 1015 of the light source 1020-a.

The wearable device 1005 may use the light propagation from the light sources 1020 to the detectors 1025 through the tissue and along the one or more optical paths for physiological measurements, such as SpO2 measurements. That is, the wearable device 1005 may use the light from the light source 1020-a, which may include red and infrared wavelengths, to measure SpO2. In some cases, SpO2 measurements may be affected by losses of in-coupled light and disturbance due to the oxygen saturation levels of blood being calculated as a ratio of two signals measured with two different wavelengths from light source 1020-a. The SpO2 measurements may be affected by light detected at detector 1025-a that has traveled through the artery 1040. As such, the optical components 1010 may be disposed on the detector 1025-a and the light source 1020 to direct the light emission pattern 1015 towards the center of the tissue and tilt the field of view 1035 towards the light emission pattern 1015 such that an overlapping portion 1045 of the field of view 1035 and the light emission pattern 1015 is located below the arteries 1040 rather than including the arteries 1040. That is, the overlapping portion 1045 of the field of view 1035 and the light emission pattern 1015 may be located within the capillaries to accurately and efficiently perform the SpO2 measurements.

In such cases, the optical components 1010 may be used to modify the light emission pattern directionally from the light sources 1020 and/or the field of view 1035 of the detectors 1025 based on a target physiology of the user, a target type of physiological measurement (e.g., PPG, heart rate, heart rate variability, and SpO2 measurements). By optimizing the optics in the wearable device 1005, the signal quality may increase the overall efficiency of the system, thereby reducing power consumption and increasing the battery life of the wearable device 1005.

FIG. 11 shows a flowchart illustrating a method 1100 that supports optical components and assembly techniques in accordance with aspects of the present disclosure. The operations of the method 1100 may be implemented by a manufacturing system or its components as described herein. For example, the operations of the method 1100 may be performed by a manufacturing system as described with reference to FIGS. 1 through 10. In some examples, a manufacturing system may execute a set of instructions to control the functional elements of the wearable device to perform the described functions. Additionally, or alternatively, the manufacturing system may perform aspects of the described functions using special-purpose hardware.

At 1105, the method may include assembling the plurality of optical components onto an inner circumference of an inner housing member of the wearable ring device and aligned with a plurality of apertures of the inner housing member of the wearable ring device, wherein the plurality of optical components comprises a first type of optical components configured to manipulate a light emission direction or a light detection direction and a second type of optical components configured to at least partially seal the plurality of apertures. The operations of block 1105 may be performed in accordance with examples as disclosed herein.

At 1110, the method may include positioning a plurality of optoelectronic components affixed to a flexible printed circuit board onto the inner circumference of the inner housing member of the wearable ring device, wherein the plurality of optoelectronic components comprise a set of light emitting components and a set of light detecting components. The operations of block 1110 may be performed in accordance with examples as disclosed herein.

At 1115, the method may include adhering the plurality of optoelectronic components to the plurality of optical components such that the plurality of optical components are aligned with the plurality of optoelectronic components and the first type of optical components are positioned between the plurality of optoelectronic components and the second type of optical components. The operations of block 1115 may be performed in accordance with examples as disclosed herein.

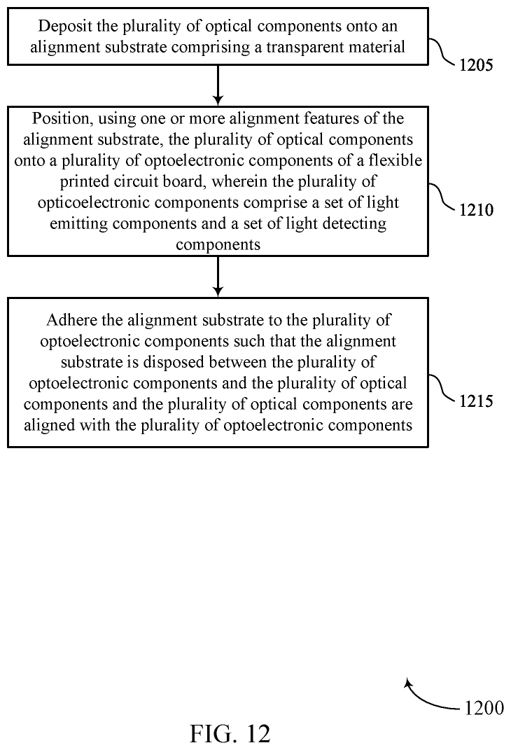

FIG. 12 illustrates a flowchart showing a method 1200 that supports array optics and fabrication in accordance with aspects of the present disclosure. The operations of the method 1200 may be implemented by a user device or its components as described herein. For example, the operations of the method 1200 may be performed by a user device as described with reference to FIGS. 1 through 10. In some examples, a user device may execute a set of instructions to control the functional elements of the user device to perform the described functions. Additionally, or alternatively, the user device may perform aspects of the described functions using special-purpose hardware.

At 1205, the method may include depositing the plurality of optical components onto an alignment substrate comprising a transparent material. The operations of 1205 may be performed in accordance with examples as disclosed herein.

At 1210, the method may include positioning, using one or more alignments features of the alignment substrate, the plurality of optical components onto a plurality of optoelectronic components of a flexible printed circuit board, wherein the plurality of optoelectronic components comprise a set of light emitting components and a set of light detecting components. The operations of 1210 may be performed in accordance with examples as disclosed herein.

At 1215, the method may include adhering the alignment substrate to the plurality of optoelectronic components such that the alignment substrate is disposed between the plurality of optoelectronic components and the plurality of optical components and the plurality of optical components are aligned with the plurality of optoelectronic components. The operations of 1215 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

A method is described. The method may include assembling the plurality of optical components onto an inner circumference of an inner housing member of the wearable ring device and aligned with a plurality of apertures of the inner housing member of the wearable ring device, wherein the plurality of optical components comprises a first type of optical components configured to manipulate a light emission direction or a light detection direction and a second type of optical components configured to at least partially seal the plurality of apertures, positioning a plurality of optoelectronic components affixed to a flexible printed circuit board onto the inner circumference of the inner housing member of the wearable ring device, wherein the plurality of optoelectronic components comprise a set of light emitting components and a set of light detecting components, and adhering the plurality of optoelectronic components to the plurality of optical components such that the plurality of optical components are aligned with the plurality of optoelectronic components and the first type of optical components are positioned between the plurality of optoelectronic components and the second type of optical components.

An apparatus is described. The apparatus may include one or more memories storing processor executable code, and one or more processors coupled with the one or more memories. The one or more processors may individually or collectively operable to execute the code to cause the apparatus to assemble the plurality of optical components onto an inner circumference of an inner housing member of the wearable ring device and aligned with a plurality of apertures of the inner housing member of the wearable ring device, wherein the plurality of optical components comprises a first type of optical components configured to manipulate a light emission direction or a light detection direction and a second type of optical components configured to at least partially seal the plurality of apertures, position a plurality of optoelectronic components affixed to a flexible printed circuit board onto the inner circumference of the inner housing member of the wearable ring device, wherein the plurality of optoelectronic components comprise a set of light emitting components and a set of light detecting components, and adhere the plurality of optoelectronic components to the plurality of optical components such that the plurality of optical components are aligned with the plurality of optoelectronic components and the first type of optical components are positioned between the plurality of optoelectronic components and the second type of optical components.

Another apparatus is described. The apparatus may include means for assembling the plurality of optical components onto an inner circumference of an inner housing member of the wearable ring device and aligned with a plurality of apertures of the inner housing member of the wearable ring device, wherein the plurality of optical components comprises a first type of optical components configured to manipulate a light emission direction or a light detection direction and a second type of optical components configured to at least partially seal the plurality of apertures, means for positioning a plurality of optoelectronic components affixed to a flexible printed circuit board onto the inner circumference of the inner housing member of the wearable ring device, wherein the plurality of optoelectronic components comprise a set of light emitting components and a set of light detecting components, and means for adhering the plurality of optoelectronic components to the plurality of optical components such that the plurality of optical components are aligned with the plurality of optoelectronic components and the first type of optical components are positioned between the plurality of optoelectronic components and the second type of optical components.

A non-transitory computer-readable medium storing code is described. The code may include instructions executable by a processor to assemble the plurality of optical components onto an inner circumference of an inner housing member of the wearable ring device and aligned with a plurality of apertures of the inner housing member of the wearable ring device, wherein the plurality of optical components comprises a first type of optical components configured to manipulate a light emission direction or a light detection direction and a second type of optical components configured to at least partially seal the plurality of apertures, position a plurality of optoelectronic components affixed to a flexible printed circuit board onto the inner circumference of the inner housing member of the wearable ring device, wherein the plurality of optoelectronic components comprise a set of light emitting components and a set of light detecting components, and adhere the plurality of optoelectronic components to the plurality of optical components such that the plurality of optical components are aligned with the plurality of optoelectronic components and the first type of optical components are positioned between the plurality of optoelectronic components and the second type of optical components.

Some examples of the method, apparatus, and non-transitory computer-readable medium described herein may further include operations, features, means, or instructions for depositing the plurality of optical components onto an alignment substrate prior to assembling the plurality of optical components onto the inner circumference of the inner housing member of the wearable ring device.

Some examples of the method, apparatus, and non-transitory computer-readable medium described herein may further include operations, features, means, or instructions for assembling the plurality of optical components onto the inner housing member occurs prior to adhering the plurality of optoelectronic components to the plurality of optical components.

Some examples of the method, apparatus, and non-transitory computer-readable medium described herein may further include operations, features, means, or instructions for applying a light source to the plurality of optical components to create a seal between the plurality of optical components and the plurality of apertures, wherein the light source may be applied prior to adhering the plurality of optoelectronic components to the plurality of optical components.

In some examples of the method, apparatus, and non-transitory computer-readable medium described herein, assembling the plurality of optical components onto the inner housing member may include operations, features, means, or instructions for depositing the first type of optical components of the plurality of optical components onto an alignment substrate and assembling the first type of optical components deposited on the alignment substrate onto the inner circumference of the inner housing member of the wearable ring device.

In some examples of the method, apparatus, and non-transitory computer-readable medium described herein, assembling the plurality of optical components onto the inner housing member may include operations, features, means, or instructions for assembling the second type of optical components of the plurality of optical components onto the inner circumference of the inner housing member of the wearable ring device after assembling the first type of optical components onto the inner circumference of the inner housing member.

Some examples of the method, apparatus, and non-transitory computer-readable medium described herein may further include operations, features, means, or instructions for applying a light source to the plurality of optical components to create a seal between the plurality of optical components and the plurality of apertures, wherein the light source may be applied prior to adhering the plurality of optoelectronic components to the plurality of optical components.

In some examples of the method, apparatus, and non-transitory computer-readable medium described herein, assembling the plurality of optical components onto the inner housing member may include operations, features, means, or instructions for assembling the second type of optical components of the plurality of optical components onto the inner circumference of the inner housing member of the wearable ring device.

Some examples of the method, apparatus, and non-transitory computer-readable medium described herein may further include operations, features, means, or instructions for applying a light source to the second type of optical components to create a seal between the second type of optical components and the plurality of apertures, wherein the light source may be applied prior to assembling the first type of optical components onto the inner circumference of the inner housing member of the wearable ring device.

Some examples of the method, apparatus, and non-transitory computer-readable medium described herein may further include operations, features, means, or instructions for depositing the first type of optical components of the plurality of optical components onto an alignment substrate and assembling the first type of optical components deposited on the alignment substrate onto the inner circumference of the inner housing member of the wearable ring device after assembling the second type of optical components onto the inner housing member.

In some examples of the method, apparatus, and non-transitory computer-readable medium described herein, the plurality of optical components comprises a lens, a reflector, a prism, a microprism, a micro lenses array, an angular filter, a metamaterial lens, a metallic material, or a combination thereof.

An apparatus is described. The apparatus may include a plurality of optical components that comprise a first type of optical component and a second type of optical component assembled onto an inner circumference of an inner housing member of the wearable ring device, wherein the plurality of optical components are aligned with a plurality of apertures of the inner housing member of the wearable ring device, wherein the first type of optical components are configured to manipulate a light emission direction or a light detection direction and the second type of optical components are configured to at least partially seal the plurality of apertures and a plurality of optoelectronic components that comprise a set of light emitting components and a set of light detecting components positioned on a flexible printed circuit board, wherein the plurality of optoelectronic components are aligned with the plurality of optical components and the first type of optical components are positioned between the plurality of optoelectronic components and the second type of optical components.

In some examples, the set of light detecting components comprise a field of view, the plurality of optical components may be configured to modify a set of characteristics of the field of view, and the set of characteristics of the field of view comprises the light detection direction, a light direction tilt angle, a light detection size, a light detection shape, or a combination thereof.

In some examples, the set of light emitting components comprise a light emission pattern, the plurality of optical components may be configured to modify a set of characteristics of the light emission pattern, and the set of characteristics of the light emission pattern comprises the light emission direction, a light emission tilt angle, a light emission size, a light emission shape, or a combination thereof.

In some examples, the plurality of optical components may be configured to adjust an overlapping portion of the field of view and the light emission pattern to be within a threshold distance from the set of light detecting components.

In some examples, the overlapping portion of the field of view and the light emission pattern may be configured to target a physiological structure within a user wearing the wearable ring device.

In some examples, the apparatus may include an alignment substrate comprising an adhesive material, wherein the plurality of optical components may be deposited on the alignment substrate.

In some examples, the first type of optical components may be aligned with the second type of optical components.

In some examples, the plurality of optical components comprise a lens, a reflector, a prism, a microprism, a micro lenses array, an angular filter, a metamaterial component, a metallic material, or a combination thereof.

In some examples, the apparatus may include a first seal between the plurality of optical components and the plurality of apertures and a second seal between the plurality of optical components and the plurality of optoelectronic components.

A method is described. The method may include depositing the plurality of optical components onto an alignment substrate comprising a transparent material, positioning, using one or more alignments features of the alignment substrate, the plurality of optical components onto a plurality of optoelectronic components of a flexible printed circuit board, wherein the plurality of optoelectronic components comprise a set of light emitting components and a set of light detecting components, and adhering the alignment substrate to the plurality of optoelectronic components such that the alignment substrate is disposed between the plurality of optoelectronic components and the plurality of optical components and the plurality of optical components are aligned with the plurality of optoelectronic components.

An apparatus is described. The apparatus may include a processor, memory coupled with the processor, and instructions stored in the memory. The instructions may be executable by the processor to cause the apparatus to deposit the plurality of optical components onto an alignment substrate comprising a transparent material, position, using one or more alignments features of the alignment substrate, the plurality of optical components onto a plurality of optoelectronic components of a flexible printed circuit board, wherein the plurality of optoelectronic components comprise a set of light emitting components and a set of light detecting components, and adhere the alignment substrate to the plurality of optoelectronic components such that the alignment substrate is disposed between the plurality of optoelectronic components and the plurality of optical components and the plurality of optical components are aligned with the plurality of optoelectronic components.

Another apparatus is described. The apparatus may include means for depositing the plurality of optical components onto an alignment substrate comprising a transparent material, means for positioning, using one or more alignments features of the alignment substrate, the plurality of optical components onto a plurality of optoelectronic components of a flexible printed circuit board, wherein the plurality of optoelectronic components comprise a set of light emitting components and a set of light detecting components, and means for adhering the alignment substrate to the plurality of optoelectronic components such that the alignment substrate is disposed between the plurality of optoelectronic components and the plurality of optical components and the plurality of optical components are aligned with the plurality of optoelectronic components.

A non-transitory computer-readable medium storing code is described. The code may include instructions executable by a processor to deposit the plurality of optical components onto an alignment substrate comprising a transparent material, positioning, using one or more alignments features of the alignment substrate, the plurality of optical components onto a plurality of optoelectronic components of a flexible printed circuit board, wherein the plurality of optoelectronic components comprise a set of light emitting components and a set of light detecting components, and adhere the alignment substrate to the plurality of optoelectronic components such that the alignment substrate is disposed between the plurality of optoelectronic components and the plurality of optical components and the plurality of optical components are aligned with the plurality of optoelectronic components.

In some examples of the method, apparatuses, and non-transitory computer-readable medium described herein, depositing the plurality of optical components onto the alignment substrate may include operations, features, means, or instructions for depositing a material of the plurality of optical components along the alignment substrate, positioning a portion of the alignment substrate through a mold based at least in part on depositing the material, and forming a shape of the plurality of optical components based at least in part on positioning the portion of the alignment substrate through the mold.

In some examples of the method, apparatuses, and non-transitory computer-readable medium described herein, depositing the plurality of optical components onto an alignment substrate may include operations, features, means, or instructions for positioning a portion of the alignment substrate through a mold, injecting a material of the plurality of optical components into the mold, and forming a shape of the plurality of optical components based at least in part on injecting the material into the mold.

Some examples of the method, apparatuses, and non-transitory computer-readable medium described herein may further include operations, features, means, or instructions for applying a light source to the plurality of optical components to create a seal between the plurality of optical components and the alignment substrate.

Some examples of the method, apparatuses, and non-transitory computer-readable medium described herein may further include operations, features, means, or instructions for applying a light source to the plurality of optoelectronic components to create a seal between the plurality of optoelectronic components and the plurality of optical components based at least in part on adhering the alignment substrate to the plurality of optoelectronic components.

Some examples of the method, apparatuses, and non-transitory computer-readable medium described herein may further include operations, features, means, or instructions for perforating the alignment substrate based at least in part on depositing the plurality of optical components onto the alignment substrate and removing a non-adhered portion of the alignment substrate from the flexible printed circuit board.

In some examples of the method, apparatuses, and non-transitory computer-readable medium described herein, positioning the plurality of optical components onto the plurality of optoelectronic components may include operations, features, means, or instructions for positioning the alignment substrate into an alignment jig comprising a first arm and a second arm such that the alignment substrate may be aligned with the flexible printed circuit board within the alignment jig.

Some examples of the method, apparatuses, and non-transitory computer-readable medium described herein may further include operations, features, means, or instructions for aligning a panel of a plurality of alignment substrates onto a panel comprising a plurality of the plurality of optoelectronic components.

Some examples of the method, apparatuses, and non-transitory computer-readable medium described herein may further include operations, features, means, or instructions for assembling the alignment substrate onto an inner circumference of an inner housing member of a wearable ring device prior to adhering the alignment substrate to the plurality of optoelectronic components.

Some examples of the method, apparatuses, and non-transitory computer-readable medium described herein may further include operations, features, means, or instructions for injecting a fillable material through an opening in the wearable ring device such that the fillable material contacts the flexible printed circuit board.

In some examples of the method, apparatuses, and non-transitory computer-readable medium described herein, the fillable material comprises an opaque material.

In some examples of the method, apparatuses, and non-transitory computer-readable medium described herein, the alignment substrate comprises an adhesive material.

In some examples of the method, apparatuses, and non-transitory computer-readable medium described herein, an optical shape of the set of light emitting components may be different than an optical shape of the set of light detecting components.

In some examples of the method, apparatuses, and non-transitory computer-readable medium described herein, the plurality of optical components comprise a reflective material.

An apparatus is described. The apparatus may include a plurality of optoelectronic components comprising a set of light emitting components and a set of light detecting components positioned on a flexible printed circuit board, a plurality of optical components adhered to the plurality of optoelectronic components, and at least a portion of an alignment substrate comprising a transparent material, wherein the alignment substrate is disposed between the plurality of optoelectronic components and the plurality of optical components.

In some examples, the alignment substrate comprises an adhesive material.

In some examples, an optical shape of the set of light emitting components is different than an optical shape of the set of light detecting components.

In some examples, the plurality of optical components comprise a reflective material.

In some examples, the apparatus may include a layer of opaque material disposed on a surface of a housing configured to house the plurality of optoelectronic components.

In some examples, a shape of the plurality of optical components aligns with a shape of the plurality of optoelectronic components.

In some examples, the apparatus may include a first seal between the plurality of optoelectronic components and the plurality of optical components and a second seal between the plurality of optical components and the alignment substrate.

In some examples, the alignment substrate comprises a perforation that circumscribes the plurality of optical components.

In some examples, the flexible printed circuit board comprises an adhesive material.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method of manufacturing a wearable ring device comprising a plurality of optical components, comprising:
assembling the plurality of optical components onto an inner circumference of an inner housing member of the wearable ring device and aligned with a plurality of apertures of the inner housing member of the wearable ring device, wherein the plurality of optical components comprises a first type of optical components configured to manipulate a light emission direction or a light detection direction and a second type of optical components configured to at least partially seal the plurality of apertures;
positioning a plurality of optoelectronic components affixed to a flexible printed circuit board onto the inner circumference of the inner housing member of the wearable ring device, wherein the plurality of optoelectronic components comprise a set of light emitting components and a set of light detecting components; and
adhering the plurality of optoelectronic components to the plurality of optical components such that the plurality of optical components are aligned with the plurality of optoelectronic components and the first type of optical components are positioned between the plurality of optoelectronic components and the second type of optical components.

2. The method of claim 1, further comprising:
depositing the plurality of optical components onto an alignment substrate prior to assembling the plurality of optical components onto the inner circumference of the inner housing member of the wearable ring device.

3. The method of claim 1, wherein assembling the plurality of optical components onto the inner housing member occurs prior to adhering the plurality of optoelectronic components to the plurality of optical components.

4. The method of claim 1, further comprising:
applying a light source to the plurality of optical components to create a seal between the plurality of optical components and the plurality of apertures, wherein the light source is applied prior to adhering the plurality of optoelectronic components to the plurality of optical components.

5. The method of claim 1, wherein assembling the plurality of optical components onto the inner housing member further comprises:
depositing the first type of optical components of the plurality of optical components onto an alignment substrate; and
assembling the first type of optical components deposited on the alignment substrate onto the inner circumference of the inner housing member of the wearable ring device.

6. The method of claim 5, wherein assembling the plurality of optical components onto the inner housing member further comprises:
assembling the second type of optical components of the plurality of optical components onto the inner circumference of the inner housing member of the wearable ring device after assembling the first type of optical components onto the inner circumference of the inner housing member.

7. The method of claim 6, further comprising:
applying a light source to the plurality of optical components to create a seal between the plurality of optical components and the plurality of apertures, wherein the light source is applied prior to adhering the plurality of optoelectronic components to the plurality of optical components.

8. The method of claim 1, wherein assembling the plurality of optical components onto the inner housing member further comprises:
assembling the second type of optical components of the plurality of optical components onto the inner circumference of the inner housing member of the wearable ring device.

9. The method of claim 8, further comprising:
applying a light source to the second type of optical components to create a seal between the second type of optical components and the plurality of apertures, wherein the light source is applied prior to assembling the first type of optical components onto the inner circumference of the inner housing member of the wearable ring device.

10. The method of claim 9, further comprising:
depositing the first type of optical components of the plurality of optical components onto an alignment substrate; and
assembling the first type of optical components deposited on the alignment substrate onto the inner circumference of the inner housing member of the wearable ring device after assembling the second type of optical components onto the inner housing member.

11. The method of claim 1, wherein the plurality of optical components comprises a lens, a reflector, a prism, a microprism, a micro lenses array, an angular filter, a metamaterial lens, a metallic material, or a combination thereof.

12. A wearable ring device, comprising:
a plurality of optical components comprising a first type of optical component and a second type of optical component assembled onto an inner circumference of an inner housing member of the wearable ring device, wherein the plurality of optical components are aligned with a plurality of apertures of the inner housing member of the wearable ring device, wherein the first type of optical components are configured to manipulate a light emission direction or a light detection direction and the second type of optical components are configured to at least partially seal the plurality of apertures; and
a plurality of optoelectronic components comprising a set of light emitting components and a set of light detecting components positioned on a flexible printed circuit board, wherein the plurality of optoelectronic components are aligned with the plurality of optical components and the first type of optical components are positioned between the plurality of optoelectronic components and the second type of optical components.

13. The wearable ring device of claim 12, wherein
the set of light detecting components comprises a field of view,
the plurality of optical components are configured to modify a set of characteristics of the field of view,
the set of characteristics of the field of view comprises the light detection direction, a light direction tilt angle, a light detection size, a light detection shape, or a combination thereof.

14. The wearable ring device of claim 13, wherein
the set of light emitting components comprises a light emission pattern,
the plurality of optical components are configured to modify a set of characteristics of the light emission pattern,
the set of characteristics of the light emission pattern comprises the light emission direction, a light emission tilt angle, a light emission size, a light emission shape, or a combination thereof.

15. The wearable ring device of claim 14, wherein the plurality of optical components are configured to adjust an overlapping portion of the field of view and the light emission pattern to be within a threshold distance from the set of light detecting components.

16. The wearable ring device of claim 15, wherein the overlapping portion of the field of view and the light emission pattern is configured to target a physiological structure within a user wearing the wearable ring device.

17. The wearable ring device of claim 12, further comprising:
an alignment substrate comprising an adhesive material, wherein the plurality of optical components are deposited on the alignment substrate.

18. The wearable ring device of claim 12, wherein the first type of optical components are aligned with the second type of optical components.

19. The wearable ring device of claim 12, wherein the plurality of optical components comprises a lens, a reflector, a prism, a microprism, a micro lenses array, an angular filter, a metamaterial component, a metallic material, or a combination thereof.

20. The wearable ring device of claim 12, further comprising:
a first seal between the plurality of optical components and the plurality of apertures; and
a second seal between the plurality of optical components and the plurality of optoelectronic components.

21. A method of manufacturing a device comprising a plurality of optical components, comprising:
depositing the plurality of optical components onto an alignment substrate comprising a transparent material;
positioning, using one or more alignment features of the alignment substrate, the plurality of optical components onto a plurality of optoelectronic components of a flexible printed circuit board, wherein the plurality of optoelectronic components comprise a set of light emitting components and a set of light detecting components; and
adhering the alignment substrate to the plurality of optoelectronic components such that the alignment substrate is disposed between the plurality of optoelectronic components and the plurality of optical components and the plurality of optical components are aligned with the plurality of optoelectronic components.

22. The method of claim 21, wherein depositing the plurality of optical components onto the alignment substrate further comprises:
depositing a material of the plurality of optical components along the alignment substrate;
positioning a portion of the alignment substrate through a mold based at least in part on depositing the material; and
forming a shape of the plurality of optical components based at least in part on positioning the portion of the alignment substrate through the mold.

23. The method of claim 21, wherein depositing the plurality of optical components onto an alignment substrate further comprises:
positioning a portion of the alignment substrate through a mold;
injecting a material of the plurality of optical components into the mold; and
forming a shape of the plurality of optical components based at least in part on injecting the material into the mold.

24. The method of claim 21, further comprising:
applying a light source to the plurality of optical components to create a seal between the plurality of optical components and the alignment substrate.

25. The method of claim 21, further comprising:
applying a light source to the plurality of optoelectronic components to create a seal between the plurality of optoelectronic components and the plurality of optical components based at least in part on adhering the alignment substrate to the plurality of optoelectronic components.

26. The method of claim 21, further comprising:
perforating the alignment substrate based at least in part on depositing the plurality of optical components onto the alignment substrate; and
removing a non-adhered portion of the alignment substrate from the flexible printed circuit board.

27. The method of claim 21, wherein positioning the plurality of optical components onto the plurality of optoelectronic components further comprises:
positioning the alignment substrate into an alignment jig comprising a first arm and a second arm such that the alignment substrate is aligned with the flexible printed circuit board within the alignment jig.

28. The method of claim 21, further comprising:
aligning a panel of a plurality of alignment substrates onto a panel comprising a plurality of the plurality of optoelectronic components.

29. The method of claim 21, further comprising:
assembling the alignment substrate onto an inner circumference of an inner housing member of a wearable ring device prior to adhering the alignment substrate to the plurality of optoelectronic components.

30. The method of claim 29, further comprising:
injecting a fillable material through an opening in the wearable ring device such that the fillable material contacts the flexible printed circuit board.

31. The method of claim 30, wherein the fillable material comprises an opaque material.

32. The method of claim 21, wherein the alignment substrate comprises an adhesive material.

33. The method of claim 21, wherein an optical shape of the set of light emitting components is different than an optical shape of the set of light detecting components.

34. The method of claim 21, wherein the plurality of optical components comprise a reflective material.

35. A wearable ring device, comprising:
a plurality of optoelectronic components comprising a set of light emitting components and a set of light detecting components positioned on a flexible printed circuit board;
a plurality of optical components adhered to the plurality of optoelectronic components; and
at least a portion of an alignment substrate comprising a transparent material, wherein the alignment substrate is disposed between the plurality of optoelectronic components and the plurality of optical components.

36. The wearable device of claim 35, wherein the alignment substrate comprises an adhesive material.

37. The wearable device of claim 35, wherein an optical shape of the set of light emitting components is different than an optical shape of the set of light detecting components.

38. The wearable device of claim 35, wherein the plurality of optical components comprise a reflective material.

39. The wearable device of claim 35, further comprising:
a layer of opaque material disposed on a surface of a housing configured to house the plurality of optoelectronic components.

40. The wearable device of claim 35, wherein a shape of the plurality of optical components aligns with a shape of the plurality of optoelectronic components.

41. The wearable device of claim 35, further comprising:
a first seal between the plurality of optoelectronic components and the plurality of optical components; and
a second seal between the plurality of optical components and the alignment substrate.

42. The wearable device of claim 35, wherein the alignment substrate comprises a perforation that circumscribes the plurality of optical components.

43. The wearable device of claim 35, wherein the flexible printed circuit board comprises an adhesive material.
